(19) United States
(12) Patent Application Publication
(10) Pub. No.: US 2026/0311840 A1
(43) Pub. Date: Oct. 8, 2026

(54) COMPUTERIZED SYSTEMS AND METHODS FOR MILITARY OPERATIONS WHERE SENSITIVE INFORMATION IS SECURELY TRANSMITTED TO ASSIGNED USERS BASED ON AI/ML DETERMINATIONS OF USER CAPABILITIES

(71) Applicant: ROM TECHNOLOGIES, INC., Brookfield, CT (US)

(72) Inventors: Steven Mason, Las Vegas, NV (US); Daniel Posnack, Fort Lauderdale, FL (US); Peter Arn, Roxbury, CT (US); Wendy Para, Las Vegas, NV (US); S. Adam Hacking, Nashua, NH (US); Micheal Mueller, Oil City, PA (US); Joseph Guaneri, Merrick, NY (US); Jonathan Greene, Denver, CO (US)

(73) Assignee: ROM TECHNOLOGIES, INC., Brookfield, CT (US)

(21) Appl. No.: 19/709,628

(22) Filed: Jun. 16, 2026

Related U.S. Application Data
(63) Continuation of application No. 17/742,233, filed on May 11, 2022, now Pat. No. 12,658,301.
Application No. 17/742,233 is a continuation of application No. 17/741,025, filed on May 10, 2022.
Application No. 17/741,025 is a continuation-in-part of application No. 17/379,542, filed on Jul. 19, 2021, now Pat. No. 11,328,807.
Application No. 17/379,542 is a continuation of application No. 17/146,705, filed on Jan. 12, 2021, now Pat. No. 12,191,018.
Application No. 17/146,705 is a continuation-in-part of application No. 17/021,895, filed on Sep. 15, 2020, now Pat. No. 11,071,597.
(60) Provisional application No. 63/113,484, filed on Nov. 13, 2020.
(60) Provisional application No. 62/910,232, filed on Oct. 3, 2019.

(51) Int. Cl. G16H 20/30; A63B 24/00; G06Q 10/0631; G06Q 10/0639; G16H 50/30

(52) U.S. Cl. CPC G16H 20/30; A63B 24/0062; G06Q 10/063112; G06Q 10/0639; G16H 50/30; A63B 2024/0065

(57) ABSTRACT

Disclosed are systems and methods for a computerized framework that leverages artificial intelligence (AI) / machine learning (ML) mechanisms to assign selected individuals to military operations. The disclosed framework comparatively analyzes an ops sheet of a military operation and profile data related to a user(s), and automatically determines user(s) who are optimal for the operation. The determined user or users possess the physical and/or intellectual capabilities to accurately and efficiently, with respect to real-world and electronic resources, perform and complete the operation. The disclosed framework provides a computerized platform that selects users for highly specific tasks based on the users’ analyzed skill sets, and based on computerized determinations of how such users are predicted to perform using those skill sets, securely and/or confidentially provides the users access to information related to the operation.

### CROSS-REFERENCE TO RELATED APPLICATIONS

This application is a continuation of U.S. Patent Application Serial No. 17/742,233, filed May 11, 2022, which is a continuation of U.S. Patent Application Serial No. 17/741,025, filed May 10, 2022, which is a continuation-in-part (CIP) of U.S. Patent Application Serial No. 17/379,542, filed July 19, 2021, now U.S. Patent No. 11/328,807, which is a continuation of U.S. Patent Application Serial No. 17/146,705, filed on January 12, 2021, which is a CIP of U.S. Patent Application Serial No. 17/021,895, filed September 15, 2020, now U.S. Patent No. 11/071,597.

U.S. Patent Application Serial No. 17/742,233 claims the benefit of and priority from U.S. Provisional Patent Application Serial No. 63/113,484, filed November 13, 2020.

U.S. Patent Application Serial No. 17/021,895 claims the benefit of and priority from U.S. Provisional Patent Application Serial No. 62/910,232, filed October 3, 2019.

The entire disclosure of each application is incorporated herein in their entirety by reference for all purposes.

### FIELD OF THE DISCLOSURE

The present disclosure relates to artificial intelligence and/or machine learning (AI/ML) processing of a user’s intellectual, emotional and/or physical fitness capabilities related to military operations, and more particularly, to dynamically determining capable users (e.g., soldiers or agents) who are suitable for military operations, and to securely providing electronic information and/or access to devices and/or accounts of such determined users to facilitate such military operations.

### BACKGROUND

Currently, users are assigned to military operations based on the users’ skill sets, as determined by a commanding officer, the users’ division, unit, experience, and the like, or some combination thereof. However, conventional techniques lack the technical capabilities for determining the probabilities of whether the actual tasks of such operations are capable of being completed, of the extent to which such completion is possible, and of the extent to which such completion can occur.

### SUMMARY

The disclosed systems and methods provide a novel computerized framework that leverages artificial intelligence (AI) and/or machine learning (ML) decision making for purposes of assigning selected individuals to particular military operations. Rather than having individuals manually review an operation and the details related thereto (e.g., referred to as an “ops sheet”), then manually select a user(s) to perform such operation, as is conventionally performed, the disclosed systems and methods introduce AI/ML mechanisms to comparatively analyze the ops sheet and profile data related to user(s) in order to determine whether a particular user is actually capable of performing the operation (and the sub-tasks included therein).

As discussed in more detail below, the disclosed systems and methods introduce computerized mechanisms to military operations. The high level of expertise such operations require, as well as the civilian, societal and business ramifications that follow, militate a critical decision-making process and evaluation that cannot simply or effectively be performed via the “human eye.” That is, while it is generally understood that commanding officers are adept at strategizing and assigning soldiers to tasks, only actual experience with particular soldiers can provide an indication about the soldiers’ respective performances.

The disclosed systems and methods provide a computerized platform that operates via a trained AI/ML engine, which enables the automatic selection of users for highly specific and specialized tasks based not only on the users’ analyzed skill sets, but also based on computerized determinations of how such users are predicted to perform when utilizing those skill sets. Thus, the disclosed framework provides a new platform for military operations to be based, strategized, assigned and executed. Not only would this evidence a decrease in natural and computer resource expenditures, but this also may increase the expectancy in lives saved (e.g., decreased loss of life due to the users’ performing such tasks having been vetted via mechanisms not previously seen).

As such, as discussed below, upon selectively assigning an operation to a user(s) that is determined “fit” for the operation, any user can be securely and/or confidentially provided access to information related to the operations tasks (e.g., ops sheet). Such a secure abeyance of providing operation materials until adequately fit users have been identified can also improve how classified (or otherwise secure) materials are held, in that only the most qualified, physical and emotionally fit individuals can be granted access to (or provided) such securely-held information.

According to some embodiments, for purposes of this disclosure, a user can refer to a person, group of people, membership, unit, division, and the like, without departing from the scope of the instant disclosure. Moreover, in some embodiments, a person can be related to a solider, agent, or specially trained individual who is specifically trained and selected for specific tasks. While the discussion herein will focus on such types of people, it should not be construed as limiting, as one of skill in the art would readily understand that the disclosure can be expanded to other types of users (e.g., users with certain types of degrees or certifications, for example), without departing from the scope of the instant disclosure.

According to some embodiments, a method is disclosed for dynamically determining capable users for military operations, and securely providing electronic information and/or access to devices and/or accounts of such determined users.

According to some embodiments, the method involves a device receiving a request associated with a real-world task, where the request includes an electronic file. The electronic file includes information related to the real-world task wherein the real-world task includes a set of activities required to be completed. The device parses the request and identifies the file. The device then analyzes the file and determines criteria associated with each element within the set of activities, where each determined criterion corresponds to a required characteristic a user must possess to complete a respective activity associated with the real-world task. The device compiles a search query based on the determined criteria, which it then uses to search a database that includes a plurality of user profiles that include characteristics of a user. The device then determines, based on the search, a search result identifying a set of user profiles, where each identified user profile includes characteristics fulfilling with the determined criteria. By rank ordering each identified user profile according to a measure of a respective user profile’s compliance with the determined criteria, the device then ranks the set of user profiles identified within the search result. A selection of a user profile is then made from the ranked search result, whereby data associated with the real-world task is securely transmitted to an account of the user associated with the selected user profile.

In accordance with one or more embodiments, the present disclosure provides a non-transitory computer-readable storage medium for executing the above-mentioned technical steps of the disclosed framework. The non-transitory computer-readable storage medium may have tangibly stored thereon, or tangibly encoded thereon, computer readable instructions that when executed by a device, cause at least one processor to perform a method for dynamically determining capable users for military operations, and securely providing electronic information and/or access to devices and/or accounts of such determined users.

In accordance with one or more embodiments, a system is provided that comprises one or more computing devices and/or apparatuses configured to provide functionality in accordance with such embodiments. In accordance with one or more embodiments, functionality may be embodied in steps of a method performed by at least one computing device and/or apparatus. In accordance with one or more embodiments, program code (or program logic) executed by a processor(s) of a computing device to implement functionality in accordance with one or more such embodiments may be embodied in, by and/or on a non-transitory computer-readable medium.

### BRIEF DESCRIPTION OF THE DRAWINGS

The features, and advantages of the disclosure will be apparent from the following description of embodiments as illustrated in the accompanying drawings, in which reference characters refer to the same parts throughout the various views. The drawings are not necessarily to scale, emphasis instead being placed upon illustrating principles of the disclosure:

FIG. 1 generally illustrates a block diagram of an embodiment of a computer implemented system according to some embodiments of the present disclosure;

FIG. 2 generally illustrates a perspective view of an embodiment of a treatment apparatus according to some embodiments of the present disclosure;

FIG. 3 generally illustrates a perspective view of a pedal of the treatment apparatus of FIG. 2 according to some embodiments of the present disclosure;

FIG. 4 generally illustrates a perspective view of a person using the treatment apparatus of FIG. 2 according to some embodiments of the present disclosure;

FIG. 5 generally illustrates an example embodiment of an overview display of an assistant interface according to some embodiments of the present disclosure;

FIG. 6 generally illustrates an example block diagram of training a machine learning model to output, based on data pertaining to the patient, a treatment plan for the patient according to some embodiments of the present disclosure;

FIG. 7 generally illustrates an embodiment of an overview display of the assistant interface presenting recommended treatment plans and excluded treatment plans in real-time during a telemedicine session according to some embodiments of the present disclosure;

FIG. 8 generally illustrates an example embodiment of a method for optimizing a treatment plan for a user to increase a probability of the user complying with the treatment plan according to some embodiments of the present disclosure;

FIG. 9 generally illustrates an example embodiment of a method for generating a treatment plan based on a desired benefit, a desired pain level, an indication of probability of complying with a particular exercise regimen, or some combination thereof according to some embodiments of the present disclosure;

FIG. 10 generally illustrates an example embodiment of a method for controlling, based on a treatment plan, a treatment apparatus while a user uses the treatment apparatus according to some embodiments of the present disclosure;

FIG. 11 generally illustrates an example computer system according to the principles of the present disclosure;

FIG. 12 is a block diagram of an example configuration within which the systems and methods disclosed herein could be implemented according to some embodiments of the present disclosure;

FIG. 13 is a block diagram illustrating components of an exemplary system according to some embodiments of the present disclosure;

FIG. 14 illustrates an exemplary data flow according to some embodiments of the present disclosure;

FIG. 15 illustrates an exemplary data flow according to some embodiments of the present disclosure;

FIG. 16 illustrates an exemplary data flow according to some embodiments of the present disclosure;

FIG. 17 illustrates an exemplary data flow according to some embodiments of the present disclosure; and

FIG. 18 illustrates an exemplary data flow according to some embodiments of the present disclosure.

### NOTATION AND NOMENCLATURE

Various terms are used to refer to particular system components. Different companies may refer to a component by different names--this document does not intend to distinguish between components that differ in name but not function. In the following discussion and in the claims, the terms “including” and “comprising” are used in an open-ended fashion, and thus should be interpreted to mean “including, but not limited to. . . . “ Also, the term “couple” or “couples” is intended to mean either an indirect or direct connection. Thus, if a first device couples to a second device, that connection may be through a direct connection or through an indirect connection via other devices and connections.

The terminology used herein is for the purpose of describing particular example embodiments only, and is not intended to be limiting. As used herein, the singular forms “a,” “an,” and “the” may be intended to include the plural forms as well, unless the context clearly indicates otherwise. The method steps, processes, and operations described herein are not to be construed as necessarily requiring their performance in the particular order discussed or illustrated, unless specifically identified as an order of performance. It is also to be understood that additional or alternative steps may be employed.

The terms first, second, third, etc. may be used herein to describe various elements, components, regions, layers and/or sections; however, these elements, components, regions, layers and/or sections should not be limited by these terms. These terms may be only used to distinguish one element, component, region, layer, or section from another region, layer, or section. Terms such as “first,” “second,” and other numerical terms, when used herein, do not imply a sequence or order unless clearly indicated by the context. Thus, a first element, component, region, layer, or section discussed below could be termed a second element, component, region, layer, or section without departing from the teachings of the example embodiments. The phrase “at least one of,” when used with a list of items, means that different combinations of one or more of the listed items may be used, and only one item in the list may be needed. For example, “at least one of: A, B, and C” includes any of the following combinations: A, B, C, A and B, A and C, B and C, and A and B and C. In another example, the phrase “one or more” when used with a list of items means there may be one item or any suitable number of items exceeding one.

Spatially relative terms, such as “inner,” “outer,” “beneath,” “below,” “lower,” “above,” “upper,” “top,” “bottom,” “inside,” “outside,” “contained within,” “superimposing upon,” and the like, may be used herein. These spatially relative terms can be used for ease of description to describe one element’s or feature’s relationship to another element(s) or feature(s) as illustrated in the figures. The spatially relative terms may also be intended to encompass different orientations of the device in use, or operation, in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptions used herein interpreted accordingly.

A “treatment plan” may include one or more treatment protocols or exercise regimens, and each treatment protocol or exercise regimen includes one or more treatment sessions or one or more exercise sessions. Each treatment session or exercise session comprises one or more session periods or exercise periods, with each session period or exercise period including at least one exercise for treating the body part of the patient. Any suitable exercise (e.g., muscular, weight lifting, cardiovascular, therapeutic, neuromuscular, neurocognitive, meditating, yoga, stretching, etc.) may be included in a session period or an exercise period. For example, a treatment plan for post-operative rehabilitation after a knee surgery may include an initial treatment protocol or exercise regimen with twice daily stretching sessions for the first 3 days after surgery and a more intensive treatment protocol with active exercise sessions performed 4 times per day starting 4 days after surgery. A treatment plan may also include information pertaining to a medical procedure to perform on the patient, a treatment protocol for the patient using a treatment apparatus, a diet regimen for the patient, a medication regimen for the patient, a sleep regimen for the patient, additional regimens, or some combination thereof.

The terms telemedicine, telehealth, telemed, teletherapeutic, telemedicine, remote medicine, etc. may be used interchangeably herein.

The term “optimal treatment plan” may refer to optimizing a treatment plan based on a certain parameter or factors or combinations of more than one parameter or factor, such as, but not limited to, a measure of benefit which one or more exercise regimens provide to users, one or more probabilities of users complying with one or more exercise regimens, an amount, quality or other measure of sleep associated with the user, information pertaining to a diet of the user, information pertaining to an eating schedule of the user, information pertaining to an age of the user, information pertaining to a sex of the user, information pertaining to a gender of the user, an indication of a mental state of the user, information pertaining to a genetic condition of the user, information pertaining to a disease state of the user, an indication of an energy level of the user, information pertaining to a microbiome from one or more locations on or in the user (e.g., skin, scalp, digestive tract, vascular system, etc.), or some combination thereof.

As used herein, the term healthcare provider may include a medical professional (e.g., such as a doctor, a nurse, a therapist, and the like), an exercise professional (e.g., such as a coach, a trainer, a nutritionist, and the like), or another professional sharing at least one of medical and exercise attributes (e.g., such as an exercise physiologist, a physical therapist, an occupational therapist, and the like). As used herein, and without limiting the foregoing, a “healthcare provider” may be a human being, a robot, a virtual assistant, a virtual assistant in virtual and/or augmented reality, or an artificially intelligent entity, such entity including a software program, integrated software and hardware, or hardware alone.

Real-time may refer to less than or equal to 2 seconds. Near real-time may refer to any interaction of a sufficiently short time to enable two individuals to engage in a dialogue via such user interface, and will preferably but not determinatively be less than 10 seconds but greater than 2 seconds.

Any of the systems and methods described in this disclosure may be used in connection with rehabilitation. Rehabilitation may be directed at cardiac rehabilitation, rehabilitation from stroke, multiple sclerosis, Parkinson’s disease, myasthenia gravis, Alzheimer’s disease, any other neurodegenative or neuromuscular disease, a brain injury, a spinal cord injury, a spinal cord disease, a joint injury, a joint disease, post-surgical recovery, or the like. Rehabilitation can further involve muscular contraction in order to improve blood flow and lymphatic flow, engage the brain and nervous system to control and affect a traumatized area to increase the speed of healing, reverse or reduce pain (including arthralgias and myalgias), reverse or reduce stiffness, recover range of motion, encourage cardiovascular engagement to stimulate the release of pain-blocking hormones or to encourage highly oxygenated blood flow to aid in an overall feeling of well-being. Rehabilitation may be provided for individuals of average weight in reasonably good physical condition having no substantial deformities, as well as for individuals more typically in need of rehabilitation, such as those who are elderly, obese, subject to disease processes, injured and/or who have a severely limited range of motion. Unless expressly stated otherwise, is to be understood that rehabilitation includes prehabilitation (also referred to as “pre-habilitation” or “prehab”). Prehabilitation may be used as a preventative procedure or as a pre-surgical or pre-treatment procedure. Prehabilitation may include any action performed by or on a patient (or directed to be performed by or on a patient, including, without limitation, remotely or distally through telemedicine) to, without limitation, prevent or reduce a likelihood of injury (e.g., prior to the occurrence of the injury); improve recovery time subsequent to surgery; improve strength subsequent to surgery; or any of the foregoing with respect to any non-surgical clinical treatment plan to be undertaken for the purpose of ameliorating or mitigating injury, dysfunction, or other negative consequence of surgical or non-surgical treatment on any external or internal part of a patient’s body. For example, a mastectomy may require prehabilitation to strengthen muscles or muscle groups affected directly or indirectly by the mastectomy. As a further non-limiting example, the removal of an intestinal tumor, the repair of a hernia, open-heart surgery or other procedures performed on internal organs or structures, whether to repair those organs or structures, to excise them or parts of them, to treat them, etc., can require cutting through, dissecting and/or harming numerous muscles and muscle groups in or about, without limitation, the skull or face, the abdomen, the ribs and/or the thoracic cavity, as well as in or about all joints and appendages. Prehabilitation can improve a patient’s speed of recovery, measure of quality of life, level of pain, etc. in all the foregoing procedures. In one embodiment of prehabilitation, a pre-surgical procedure or a pre-non-surgical-treatment may include one or more sets of exercises for a patient to perform prior to such procedure or treatment. Performance of the one or more sets of exercises may be required in order to qualify for an elective surgery, such as a knee replacement. The patient may prepare an area of his or her body for the surgical procedure by performing the one or more sets of exercises, thereby strengthening muscle groups, improving existing muscle memory, reducing pain, reducing stiffness, establishing new muscle memory, enhancing mobility (i.e., improve range of motion), improving blood flow, and/or the like. The phrase, and all permutations of the phrase, “respective measure of benefit with which one or more exercise regimens may provide the user” (e.g., “measure of benefit,” “respective measures of benefit,” “measures of benefit,” “measure of exercise regimen benefit,” “exercise regimen benefit measurement,” etc.) may refer to one or more measures of benefit with which one or more exercise regimens may provide the user.

### DETAILED DESCRIPTION OF EXAMPLE EMBODIMENTS

The following discussion is directed to various embodiments of the present disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

The following discussion is directed to various embodiments of the present disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

Determining a treatment plan for a patient having certain characteristics (e.g., vital-sign or other measurements; performance; demographic; psychographic; geographic; diagnostic; measurement- or test-based; medically historic; behavioral historic; cognitive; etiologic; cohort-associative; differentially diagnostic; surgical, physically therapeutic, microbiome related, pharmacologic and other treatment(s) recommended; arterial blood gas and/or oxygenation levels or percentages; glucose levels; blood oxygen levels; insulin levels; psychographics; etc.) may be a technically challenging problem. For example, a multitude of information may be considered when determining a treatment plan, which may result in inefficiencies and inaccuracies in the treatment plan selection process. In a rehabilitative setting, some of the multitude of information considered may include characteristics of the patient such as personal information, performance information, and measurement information. The personal information may include, e.g., demographic, psychographic or other information, such as an age, a weight, a gender, a height, a body mass index, a medical condition, a familial medication history, an injury, a medical procedure, a medication prescribed, or some combination thereof. The performance information may include, e.g., an elapsed time of using a treatment apparatus, an amount of force exerted on a portion of the treatment apparatus, a range of motion achieved on the treatment apparatus, a movement speed of a portion of the treatment apparatus, a duration of use of the treatment apparatus, an indication of a plurality of pain levels using the treatment apparatus, or some combination thereof. The measurement information may include, e.g., a vital sign, a respiration rate, a heartrate, a temperature, a blood pressure, a glucose level, arterial blood gas and/or oxygenation levels or percentages, or other biomarker, or some combination thereof. It may be desirable to process and analyze the characteristics of a multitude of patients, the treatment plans performed for those patients, and the results of the treatment plans for those patients.

Further, another technical problem may involve distally treating, via a computing apparatus during a telemedicine session, a patient from a location different than a location at which the patient is located. An additional technical problem is controlling or enabling, from the different location, the control of a treatment apparatus used by the patient at the patient’s location. Oftentimes, when a patient undergoes rehabilitative surgery (e.g., knee surgery), a healthcare provider may prescribe a treatment apparatus to the patient to use to perform a treatment protocol at their residence or at any mobile location or temporary domicile. A healthcare provider may refer to a doctor, physician assistant, nurse, chiropractor, dentist, physical therapist, acupuncturist, physical trainer, or the like. A healthcare provider may refer to any person with a credential, license, degree, or the like in the field of medicine, physical therapy, rehabilitation, or the like.

When the healthcare provider is located in a different location from the patient and the treatment apparatus, it may be technically challenging for the healthcare provider to monitor the patient’s actual progress (as opposed to relying on the patient’s word about their progress) in using the treatment apparatus, modify the treatment plan according to the patient’s progress, adapt the treatment apparatus to the personal characteristics of the patient as the patient performs the treatment plan, and the like.

Additionally, or alternatively, a computer-implemented system may be used in connection with a treatment apparatus to treat the patient, for example, during a telemedicine session. For example, the treatment apparatus can be configured to be manipulated by a user while the user is performing a treatment plan. The system may include a patient interface that includes an output device configured to present telemedicine information associated with the telemedicine session. During the telemedicine session, the processing device can be configured to receive treatment data pertaining to the user. The treatment data may include one or more characteristics of the user. The processing device may be configured to determine, via one or more trained machine learning models, at least one respective measure of benefit which one or more exercise regimens provide the user. Determining the respective measure of benefit may be based on the treatment data. The processing device may be configured to determine, via the one or more trained machine learning models, one or more probabilities of the user complying with the one or more exercise regimens. The processing device may be configured to transmit the treatment plan, for example, to a computing device. The treatment plan can be generated based on the one or more probabilities and the respective measure of benefit which the one or more exercise regimens provide the user.

Accordingly, systems and methods, such as those described herein, that receive treatment data pertaining to the user of the treatment apparatus during telemedicine session, may be desirable.

In some embodiments, the systems and methods described herein may be configured to use a treatment apparatus configured to be manipulated by an individual while performing a treatment plan. The individual may include a user, patient, or other a person using the treatment apparatus to perform various exercises for prehabilitation, rehabilitation, stretch training, and the like. The systems and methods described herein may be configured to use and/or provide a patient interface comprising an output device configured to present telemedicine information associated with a telemedicine session.

In some embodiments, during an adaptive telemedicine session, the systems and methods described herein may be configured to use artificial intelligence and/or machine learning to assign patients to cohorts and to dynamically control a treatment apparatus based on the assignment. The term “adaptive telemedicine” may refer to a telemedicine session dynamically adapted based on one or more factors, criteria, parameters, characteristics, or the like. The one or more factors, criteria, parameters, characteristics, or the like may pertain to the user (e.g., heartrate, blood pressure, perspiration rate, pain level, or the like), the treatment apparatus (e.g., pressure, range of motion, speed of motor, etc.), details of the treatment plan, and so forth.

In some embodiments, numerous patients may be prescribed numerous treatment apparatuses because the numerous patients are recovering from the same medical procedure and/or suffering from the same injury. The numerous treatment apparatuses may be provided to the numerous patients. The treatment apparatuses may be used by the patients to perform treatment plans in their residences, at gyms, at rehabilitative centers, at hospitals, or at any suitable locations, including permanent or temporary domiciles.

In some embodiments, the treatment apparatuses may be communicatively coupled to a server. Characteristics of the patients, including the treatment data, may be collected before, during, and/or after the patients perform the treatment plans. For example, any or each of the personal information, the performance information, and the measurement information may be collected before, during, and/or after a patient performs the treatment plans. The results (e.g., improved performance or decreased performance) of performing each exercise may be collected from the treatment apparatus throughout the treatment plan and after the treatment plan is performed. The parameters, settings, configurations, etc. (e.g., position of pedal, amount of resistance, etc.) of the treatment apparatus may be collected before, during, and/or after the treatment plan is performed.

Each characteristic of the patient, each result, and each parameter, setting, configuration, etc. may be timestamped and may be correlated with a particular step or set of steps in the treatment plan. Such a technique may enable the determination of which steps in the treatment plan lead to desired results (e.g., improved muscle strength, range of motion, etc.) and which steps lead to diminishing returns (e.g., continuing to exercise after 3 minutes actually delays or harms recovery).

Data may be collected from the treatment apparatuses and/or any suitable computing device (e.g., computing devices where personal information is entered, such as the interface of the computing device described herein, a clinician interface, patient interface, or the like) over time as the patients use the treatment apparatuses to perform the various treatment plans. The data that may be collected may include the characteristics of the patients, the treatment plans performed by the patients, and the results of the treatment plans. Further, the data may include characteristics of the treatment apparatus. The characteristics of the treatment apparatus may include a make (e.g., identity of entity that designed, manufactured, etc. the treatment apparatus 70) of the treatment apparatus 70, a model (e.g., model number or other identifier of the model) of the treatment apparatus 70, a year (e.g., year the treatment apparatus was manufactured) of the treatment apparatus 70, operational parameters (e.g., engine temperature during operation, a respective status of each of one or more sensors included in or associated with the treatment apparatus 70, vibration measurements of the treatment apparatus 70 in operation, measurements of static and/or dynamic forces exerted internally or externally on the treatment apparatus 70, etc.) of the treatment apparatus 70, settings (e.g., range of motion setting, speed setting, required pedal force setting, etc.) of the treatment apparatus 70, and the like. The data collected from the treatment apparatuses, computing devices, characteristics of the user, characteristics of the treatment apparatus, and the like may be collectively referred to as “treatment data” herein.

In some embodiments, the data may be processed to group certain people into cohorts. The people may be grouped by people having certain or selected similar characteristics, treatment plans, and results of performing the treatment plans. For example, athletic people having no medical conditions who perform a treatment plan (e.g., use the treatment apparatus for 30 minutes a day 5 times a week for 3 weeks) and who fully recover may be grouped into a first cohort. Older people who are classified obese and who perform a treatment plan (e.g., use the treatment plan for 10 minutes a day 3 times a week for 4 weeks) and who improve their range of motion by 75 percent may be grouped into a second cohort.

In some embodiments, an artificial intelligence engine may include one or more machine learning models that are trained using the cohorts. In some embodiments, the artificial intelligence engine may be used to identify trends and/or patterns and to define new cohorts based on achieving desired results from the treatment plans and machine learning models associated therewith may be trained to identify such trends and/or patterns and to recommend and rank the desirability of the new cohorts. For example, the one or more machine learning models may be trained to receive an input of characteristics of a new patient and to output a treatment plan for the patient that results in a desired result. The machine learning models may match a pattern between the characteristics of the new patient and at least one patient of the patients included in a particular cohort. When a pattern is matched, the machine learning models may assign the new patient to the particular cohort and select the treatment plan associated with the at least one patient. The artificial intelligence engine may be configured to control, distally and based on the treatment plan, the treatment apparatus while the new patient uses the treatment apparatus to perform the treatment plan.

As may be appreciated, the characteristics of the new patient (e.g., a new user) may change as the new patient uses the treatment apparatus to perform the treatment plan. For example, the performance of the patient may improve quicker than expected for people in the cohort to which the new patient is currently assigned. Accordingly, the machine learning models may be trained to dynamically reassign, based on the changed characteristics, the new patient to a different cohort that includes people having characteristics similar to the now-changed characteristics as the new patient. For example, a clinically obese patient may lose weight and no longer meet the weight criterion for the initial cohort, result in the patient’s being reassigned to a different cohort with a different weight criterion.

A different treatment plan may be selected for the new patient, and the treatment apparatus may be controlled, distally (e.g., which may be referred to as remotely) and based on the different treatment plan, the treatment apparatus while the new patient uses the treatment apparatus to perform the treatment plan. Such techniques may provide the technical solution of distally controlling a treatment apparatus.

Further, the systems and methods described herein may lead to faster recovery times and/or better results for the patients because the treatment plan that most accurately fits their characteristics is selected and implemented, in real-time, at any given moment. “Real-time” may also refer to near real-time, which may be less than 10 seconds or any reasonably proximate difference between two different times. As described herein, the term “results” may refer to medical results or medical outcomes. Results and outcomes may refer to responses to medical actions. The term “medical action(s)” may refer to any suitable action performed by the healthcare provider, and such action or actions may include diagnoses, prescription of treatment plans, prescription of treatment apparatuses, and the making, composing and/or executing of appointments, telemedicine sessions, prescription of medicines, telephone calls, emails, text messages, and the like.

Depending on what result is desired, the artificial intelligence engine may be trained to output several treatment plans. For example, one result may include recovering to a threshold level (e.g., 75% range of motion) in a fastest amount of time, while another result may include fully recovering (e.g., 100% range of motion) regardless of the amount of time. The data obtained from the patients and sorted into cohorts may indicate that a first treatment plan provides the first result for people with characteristics similar to the patient’s, and that a second treatment plan provides the second result for people with characteristics similar to the patient.

Further, the artificial intelligence engine may be trained to output treatment plans that are not optimal i.e., sub-optimal, nonstandard, or otherwise excluded (all referred to, without limitation, as “excluded treatment plans”) for the patient. For example, if a patient has high blood pressure, a particular exercise may not be approved or suitable for the patient as it may put the patient at unnecessary risk or even induce a hypertensive crisis and, accordingly, that exercise may be flagged in the excluded treatment plan for the patient. In some embodiments, the artificial intelligence engine may monitor the treatment data received while the patient (e.g., the user) with, for example, high blood pressure, uses the treatment apparatus to perform an appropriate treatment plan and may modify the appropriate treatment plan to include features of an excluded treatment plan that may provide beneficial results for the patient if the treatment data indicates the patient is handling the appropriate treatment plan without aggravating, for example, the high blood pressure condition of the patient. In some embodiments, the artificial intelligence engine may modify the treatment plan if the monitored data shows the plan to be inappropriate or counterproductive for the user.

In some embodiments, the treatment plans and/or excluded treatment plans may be presented, during a telemedicine or telehealth session, to a healthcare provider. The healthcare provider may select a particular treatment plan for the patient to cause that treatment plan to be transmitted to the patient and/or to control, based on the treatment plan, the treatment apparatus. In some embodiments, to facilitate telehealth or telemedicine applications, including remote diagnoses, determination of treatment plans and rehabilitative and/or pharmacologic prescriptions, the artificial intelligence engine may receive and/or operate distally from the patient and the treatment apparatus.

In such cases, the recommended treatment plans and/or excluded treatment plans may be presented simultaneously with a video of the patient in real-time or near real-time during a telemedicine or telehealth session on a user interface of a computing apparatus of a healthcare provider. The video may also be accompanied by audio, text and other multimedia information and/or other sensorial or perceptive (e.g., tactile, gustatory, haptic, pressure-sensing-based or electromagnetic (e.g., neurostimulation). Real-time may refer to less than or equal to 2 seconds. Near real-time may refer to any interaction of a sufficiently short time to enable two individuals to engage in a dialogue via such user interface, and will generally be less than 10 seconds (or any suitably proximate difference between two different times) but greater than 2 seconds. Presenting the treatment plans generated by the artificial intelligence engine concurrently with a presentation of the patient video may provide an enhanced user interface because the healthcare provider may continue to visually and/or otherwise communicate with the patient while also reviewing the treatment plans on the same user interface. The enhanced user interface may improve the healthcare provider’s experience using the computing device and may encourage the healthcare provider to reuse the user interface. Such a technique may also reduce computing resources (e.g., processing, memory, network) because the healthcare provider does not have to switch to another user interface screen to enter a query for a treatment plan to recommend based on the characteristics of the patient. The artificial intelligence engine may be configured to provide, dynamically on the fly, the treatment plans and excluded treatment plans.

In some embodiments, the treatment plan may be modified by a healthcare provider. For example, certain procedures may be added, modified or removed. In the telehealth scenario, there are certain procedures that may not be performed due to the distal nature of a healthcare provider using a computing device in a different physical location than a patient.

A technical problem may relate to the information pertaining to the patient’s medical condition being received in disparate formats. For example, a server may receive the information pertaining to a medical condition of the patient from one or more sources (e.g., from an electronic medical record (EMR) system, application programming interface (API), or any suitable system that has information pertaining to the medical condition of the patient). That is, some sources used by various healthcare provider entities may be installed on their local computing devices and, additionally and/or alternatively, may use proprietary formats. Accordingly, some embodiments of the present disclosure may use an API to obtain, via interfaces exposed by APIs used by the sources, the formats used by the sources. In some embodiments, when information is received from the sources, the API may map and convert the format used by the sources to a standardized (i.e., canonical) format, language and/or encoding (“format” as used herein will be inclusive of all of these terms) used by the artificial intelligence engine. Further, the information converted to the standardized format used by the artificial intelligence engine may be stored in a database accessed by the artificial intelligence engine when the artificial intelligence engine is performing any of the techniques disclosed herein. Using the information converted to a standardized format may enable a more accurate determination of the procedures to perform for the patient.

The various embodiments disclosed herein may provide a technical solution to the technical problem pertaining to the patient’s medical condition information being received in disparate formats. For example, a server may receive the information pertaining to a medical condition of the patient from one or more sources (e.g., from an electronic medical record (EMR) system, application programming interface (API), or any suitable system that has information pertaining to the medical condition of the patient). The information may be converted from the format used by the sources to the standardized format used by the artificial intelligence engine. Further, the information converted to the standardized format used by the artificial intelligence engine may be stored in a database accessed by the artificial intelligence engine when performing any of the techniques disclosed herein. The standardized information may enable generating optimal treatment plans, where the generating is based on treatment plans associated with the standardized information. The optimal treatment plans may be provided in a standardized format that can be processed by various applications (e.g., telehealth) executing on various computing devices of healthcare providers and/or patients.

A technical problem may include a challenge of generating treatment plans for users, such treatment plans comprising exercises that balance a measure of benefit which the exercise regimens provide to the user and the probability the user complies with the exercises (or the distinct probabilities the user complies with each of the one or more exercises). By selecting exercises having higher compliance probabilities for the user, more efficient treatment plans may be generated, and these may enable less frequent use of the treatment apparatus and therefore extend the lifetime or time between recommended maintenance of or needed repairs to the treatment apparatus. For example, if the user consistently quits a certain exercise but yet attempts to perform the exercise multiple times thereafter, the treatment apparatus may be used more times, and therefore suffer more “wear-and-tear” than if the user fully complies with the exercise regimen the first time. In some embodiments, a technical solution may include using trained machine learning models to generate treatment plans based on the measure of benefit exercise regimens provide users and the probabilities of the users associated with complying with the exercise regimens, such inclusion thereby leading to more time-efficient, cost-efficient, and maintenance-efficient use of the treatment apparatus.

In some embodiments, the treatment apparatus may be adaptive and/or personalized because its properties, configurations, and positions may be adapted to the needs of a particular patient. For example, the pedals may be dynamically adjusted on the fly (e.g., via a telemedicine session or based on programmed configurations in response to certain measurements being detected) to increase or decrease a range of motion to comply with a treatment plan designed for the user. In some embodiments, a healthcare provider may adapt, remotely during a telemedicine session, the treatment apparatus to the needs of the patient by causing a control instruction to be transmitted from a server to treatment apparatus. Such adaptive nature may improve the results of recovery for a patient, furthering the goals of personalized medicine, and enabling personalization of the treatment plan on a per-individual basis.

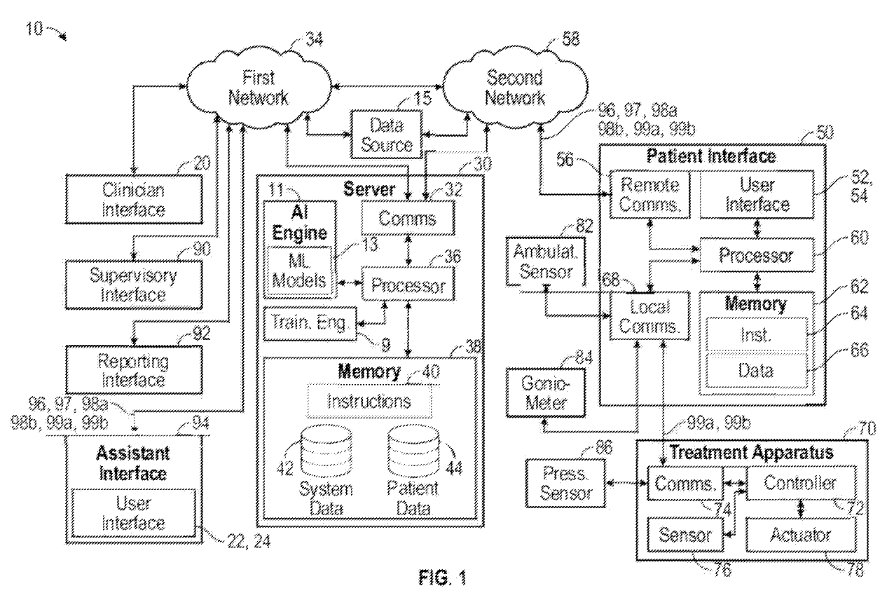

FIG. 1 shows a block diagram of a computer-implemented system 10, hereinafter called “the system” for managing a treatment plan. Managing the treatment plan may include using an artificial intelligence engine to recommend treatment plans and/or provide excluded treatment plans that should not be recommended to a patient.

The system 10 also includes a server 30 configured to store and to provide data related to managing the treatment plan. The server 30 may include one or more computers and may take the form of a distributed and/or virtualized computer or computers. The server 30 also includes a first communication interface 32 configured to communicate with the clinician interface 20 via a first network 34. In some embodiments, the first network 34 may include wired and/or wireless network connections such as Wi-Fi, Bluetooth, ZigBee, Near-Field Communications (NFC), cellular data network, etc. The server 30 includes a first processor 36 and a first machine-readable storage memory 38, which may be called a “memory” for short, holding first instructions 40 for performing the various actions of the server 30 for execution by the first processor 36. The server 30 is configured to store data regarding the treatment plan. For example, the memory 38 includes a system data store 42 configured to hold system data, such as data pertaining to treatment plans for treating one or more patients.

The system data store 42 may be configured to store optimal treatment plans generated based on one or more probabilities of users associated with complying with the exercise regimens, and the measure of benefit with which one or more exercise regimens provide the user. The system data store 42 may hold data pertaining to one or more exercises (e.g., a type of exercise, which body part the exercise affects, a duration of the exercise, which treatment apparatus to use to perform the exercise, repetitions of the exercise to perform, etc.). When any of the techniques described herein are being performed, or prior to or thereafter such performance, any of the data stored in the system data store 42 may be accessed by an artificial intelligence engine 11.

The server 30 may also be configured to store data regarding performance by a patient in following a treatment plan. For example, the memory 38 includes a patient data store 44 configured to hold patient data, such as data pertaining to the one or more patients, including data representing each patient’s performance within the treatment plan. The patient data store 44 may hold treatment data pertaining to users over time, such that historical treatment data is accumulated in the patient data store 44. The patient data store 44 may hold data pertaining to measures of benefit one or more exercises provide to users, probabilities of the users complying with the exercise regimens, and the like. The exercise regimens may include any suitable number of exercises (e.g., shoulder raises, squats, cardiovascular exercises, sit-ups, curls, etc.) to be performed by the user. When any of the techniques described herein are being performed, or prior to or thereafter such performance, any of the data stored in the patient data store 44 may be accessed by an artificial intelligence engine 11.

In addition, the determination or identification of: the characteristics (e.g., personal, performance, measurement, etc.) of the users, the treatment plans followed by the users, the measure of benefits which exercise regimens provide to the users, the probabilities of the users associated with complying with exercise regimens, the level of compliance with the treatment plans (e.g., the user completed 4 out of 5 exercises in the treatment plans, the user completed 80% of an exercise in the treatment plan, etc.), and the results of the treatment plans may use correlations and other statistical or probabilistic measures to enable the partitioning of or to partition the treatment plans into different patient cohort-equivalent databases in the patient data store 44. For example, the data for a first cohort of first patients having a first determined measure of benefit provided by exercise regimens, a first determined probability of the user associated with complying with exercise regimens, a first similar injury, a first similar medical condition, a first similar medical procedure performed, a first treatment plan followed by the first patient, and/or a first result of the treatment plan, may be stored in a first patient database. The data for a second cohort of second patients having a second determined measure of benefit provided by exercise regimens, a second determined probability of the user associated with complying with exercise regimens, a second similar injury, a second similar medical condition, a second similar medical procedure performed, a second treatment plan followed by the second patient, and/or a second result of the treatment plan may be stored in a second patient database. Any single characteristic, any combination of characteristics, or any measures calculation therefrom or thereupon may be used to separate the patients into cohorts. In some embodiments, the different cohorts of patients may be stored in different partitions or volumes of the same database. There is no specific limit to the number of different cohorts of patients allowed, other than as limited by mathematical combinatoric and/or partition theory.

This measure of exercise benefit data, user compliance probability data, characteristic data, treatment plan data, and results data may be obtained from numerous treatment apparatuses and/or computing devices over time and stored in the database 44. The measure of exercise benefit data, user compliance probability data, characteristic data, treatment plan data, and results data may be correlated in the patient-cohort databases in the patient data store 44. The characteristics of the users may include personal information, performance information, and/or measurement information.

In addition to the historical treatment data, measure of exercise benefit data, and/or user compliance probability data about other users stored in the patient cohort-equivalent databases, real-time or near-real-time information based on the current patient’s treatment data, measure of exercise benefit data, and/or user compliance probability data about a current patient being treated may be stored in an appropriate patient cohort-equivalent database. The treatment data, measure of exercise benefit data, and/or user compliance probability data of the patient may be determined to match or be similar to the treatment data, measure of exercise benefit data, and/or user compliance probability data of another person in a particular cohort (e.g., a first cohort “A”, a second cohort “B” or a third cohort “C”, etc.) and the patient may be assigned to the selected or associated cohort.

In some embodiments, the server 30 may execute the artificial intelligence (AI) engine 11 that uses one or more machine learning models 13 to perform at least one of the embodiments disclosed herein. The server 30 may include a training engine 9 capable of generating the one or more machine learning models 13. The machine learning models 13 may be trained to assign users to certain cohorts based on their treatment data, generate treatment plans using real-time and historical data correlations involving patient cohort-equivalents, and control a treatment apparatus 70, among other things. The machine learning models 13 may be trained to generate, based on one or more probabilities of the user complying with one or more exercise regimens and/or a respective measure of benefit one or more exercise regimens provide the user, a treatment plan at least a subset of the one or more exercises for the user to perform. The one or more machine learning models 13 may be generated by the training engine 9 and may be implemented in computer instructions executable by one or more processing devices of the training engine 9 and/or the servers 30. To generate the one or more machine learning models 13, the training engine 9 may train the one or more machine learning models 13. The one or more machine learning models 13 may be used by the artificial intelligence engine 11.

The training engine 9 may be a rackmount server, a router computer, a personal computer, a portable digital assistant, a smartphone, a laptop computer, a tablet computer, a netbook, a desktop computer, an Internet of Things (IoT) device, any other desired computing device, or any combination of the above. The training engine 9 may be cloud-based or a real-time software platform, and it may include privacy software or protocols, and/or security software or protocols.

To train the one or more machine learning models 13, the training engine 9 may use a training data set of a corpus of information (e.g., treatment data, measures of benefits of exercises provide to users, probabilities of users complying with the one or more exercise regimens, etc.) pertaining to users who performed treatment plans using the treatment apparatus 70, the details (e.g., treatment protocol including exercises, amount of time to perform the exercises, instructions for the patient to follow, how often to perform the exercises, a schedule of exercises, parameters/configurations/settings of the treatment apparatus 70 throughout each step of the treatment plan, etc.) of the treatment plans performed by the users using the treatment apparatus 70, and/or the results of the treatment plans performed by the users, etc.

The one or more machine learning models 13 may be trained to match patterns of treatment data of a user with treatment data of other users assigned to a particular cohort. The term “match” may refer to an exact match, a correlative match, a substantial match, a probabilistic match, etc. The one or more machine learning models 13 may be trained to receive the treatment data of a patient as input, map the treatment data to the treatment data of users assigned to a cohort, and determine a respective measure of benefit one or more exercise regimens provide to the user based on the measures of benefit the exercises provided to the users assigned to the cohort. The one or more machine learning models 13 may be trained to receive the treatment data of a patient as input, map the treatment data to treatment data of users assigned to a cohort, and determine one or more probabilities of the user associated with complying with the one or more exercise regimens based on the probabilities of the users in the cohort associated with complying with the one or more exercise regimens. The one or more machine learning models 13 may also be trained to receive various input (e.g., the respective measure of benefit which one or more exercise regimens provide the user; the one or more probabilities of the user complying with the one or more exercise regimens; an amount, quality or other measure of sleep associated with the user; information pertaining to a diet of the user, information pertaining to an eating schedule of the user; information pertaining to an age of the user, information pertaining to a sex of the user; information pertaining to a gender of the user; an indication of a mental state of the user; information pertaining to a genetic condition of the user; information pertaining to a disease state of the user; an indication of an energy level of the user; or some combination thereof), and to output a generated treatment plan for the patient.

The one or more machine learning models 13 may be trained to match patterns of a first set of parameters (e.g., treatment data, measures of benefits of exercises provided to users, probabilities of user compliance associated with the exercises, etc.) with a second set of parameters associated with an optimal treatment plan. The one or more machine learning models 13 may be trained to receive the first set of parameters as input, map the characteristics to the second set of parameters associated with the optimal treatment plan, and select the optimal treatment plan. The one or more machine learning models 13 may also be trained to control, based on the treatment plan, the treatment apparatus 70.

Using training data that includes training inputs and corresponding target outputs, the one or more machine learning models 13 may refer to model artifacts created by the training engine 9. The training engine 9 may find patterns in the training data wherein such patterns map the training input to the target output, and generate the machine learning models 13 that capture these patterns. In some embodiments, the artificial intelligence engine 11, the database 33, and/or the training engine 9 may reside on another component (e.g., assistant interface 94, clinician interface 20, etc.) depicted in FIG. 1.

The one or more machine learning models 13 may comprise, e.g., a single level of linear or non-linear operations (e.g., a support vector machine (SVM)) or the machine learning models 13 may be a deep network, i.e., a machine learning model comprising multiple levels of non-linear operations. Examples of deep networks are neural networks including generative adversarial networks, convolutional neural networks, recurrent neural networks with one or more hidden layers, and fully connected neural networks (e.g., each neuron may transmit its output signal to the input of the remaining neurons, as well as to itself). For example, the machine learning model may include numerous layers and/or hidden layers that perform calculations (e.g., dot products) using various neurons.

Further, in some embodiments, based on subsequent data (e.g., treatment data, measures of exercise benefit data, probabilities of user compliance data, treatment plan result data, etc.) received, the machine learning models 13 may be continuously or continually updated. For example, the machine learning models 13 may include one or more hidden layers, weights, nodes, parameters, and the like. As the subsequent data is received, the machine learning models 13 may be updated such that the one or more hidden layers, weights, nodes, parameters, and the like are updated to match or be computable from patterns found in the subsequent data. Accordingly, the machine learning models 13 may be re-trained on the fly as subsequent data is received, and therefore, the machine learning models 13 may continue to learn.

The system 10 also includes a patient interface 50 configured to communicate information to a patient and to receive feedback from the patient. Specifically, the patient interface includes an input device 52 and an output device 54, which may be collectively called a patient user interface 52, 54. The input device 52 may include one or more devices, such as a keyboard, a mouse, a touch screen input, a gesture sensor, and/or a microphone and processor configured for voice recognition. The output device 54 may take one or more different forms including, for example, a computer monitor or display screen on a tablet, smartphone, or a smart watch. The output device 54 may include other hardware and/or software components such as a projector, virtual reality capability, augmented reality capability, etc. The output device 54 may incorporate various different visual, audio, or other presentation technologies. For example, the output device 54 may include a non-visual display, such as an audio signal, which may include spoken language and/or other sounds such as tones, chimes, and/or melodies, which may signal different conditions and/or directions and/or other sensorial or perceptive (e.g., tactile, gustatory, haptic, pressure-sensing-based or electromagnetic (e.g., neurostimulation) communication devices. The output device 54 may comprise one or more different display screens presenting various data and/or interfaces or controls for use by the patient. The output device 54 may include graphics, which may be presented by a web-based interface and/or by a computer program or application (App.). In some embodiments, the patient interface 50 may include functionality provided by or similar to existing voice-based assistants such as Siri by Apple, Alexa by Amazon, Google Assistant, or Bixby by Samsung.

In some embodiments, the output device 54 may present a user interface that may present a recommended treatment plan, excluded treatment plan, or the like to the patient. The user interface may include one or more graphical elements that enable the user to select which treatment plan to perform. Responsive to receiving a selection of a graphical element (e.g., “Start” button) associated with a treatment plan via the input device 54, the patient interface 50 may communicate a control signal to the controller 72 of the treatment apparatus, wherein the control signal causes the treatment apparatus 70 to begin execution of the selected treatment plan. As described below, the control signal may control, based on the selected treatment plan, the treatment apparatus 70 by causing actuation of the actuator 78 (e.g., cause a motor to drive rotation of pedals of the treatment apparatus at a certain speed), causing measurements to be obtained via the sensor 76, or the like. The patient interface 50 may communicate, via a local communication interface 68, the control signal to the treatment apparatus 70.

As shown in FIG. 1, the patient interface 50 includes a second communication interface 56, which may also be called a remote communication interface configured to communicate with the server 30 and/or the clinician interface 20 via a second network 58. In some embodiments, the second network 58 may include a local area network (LAN), such as an Ethernet network. In some embodiments, the second network 58 may include the Internet, and communications between the patient interface 50 and the server 30 and/or the clinician interface 20 may be secured via encryption, such as, for example, by using a virtual private network (VPN). In some embodiments, the second network 58 may include wired and/or wireless network connections such as Wi-Fi, Bluetooth, ZigBee, Near-Field Communications (NFC), cellular data network, etc. In some embodiments, the second network 58 may be the same as and/or operationally coupled to the first network 34.

The patient interface 50 includes a second processor 60 and a second machine-readable storage memory 62 holding second instructions 64 for execution by the second processor 60 for performing various actions of patient interface 50. The second machine-readable storage memory 62 also includes a local data store 66 configured to hold data, such as data pertaining to a treatment plan and/or patient data, such as data representing a patient’s performance within a treatment plan. The patient interface 50 also includes a local communication interface 68 configured to communicate with various devices for use by the patient in the vicinity of the patient interface 50. The local communication interface 68 may include wired and/or wireless communications. In some embodiments, the local communication interface 68 may include a local wireless network such as Wi-Fi, Bluetooth, ZigBee, Near-Field Communications (NFC), cellular data network, etc.

The system 10 also includes a treatment apparatus 70 configured to be manipulated by the patient and/or to manipulate a body part of the patient for performing activities according to the treatment plan. In some embodiments, the treatment apparatus 70 may take the form of an exercise and rehabilitation apparatus configured to perform and/or to aid in the performance of a rehabilitation regimen, which may be an orthopedic rehabilitation regimen, and the treatment includes rehabilitation of a body part of the patient, such as a joint or a bone or a muscle group. The treatment apparatus 70 may be any suitable medical, rehabilitative, therapeutic, etc. apparatus configured to be controlled distally via another computing device to treat a patient and/or exercise the patient. The treatment apparatus 70 may be an electromechanical machine including one or more weights, an electromechanical bicycle, an electromechanical spin-wheel, a smart-mirror, a treadmill, or the like. The body part may include, for example, a spine, a hand, a foot, a knee, or a shoulder. The body part may include a part of a joint, a bone, or a muscle group, such as one or more vertebrae, a tendon, or a ligament. As shown in FIG. 1, the treatment apparatus 70 includes a controller 72, which may include one or more processors, computer memory, and/or other components. The treatment apparatus 70 also includes a fourth communication interface 74 configured to communicate with the patient interface 50 via the local communication interface 68. The treatment apparatus 70 also includes one or more internal sensors 76 and an actuator 78, such as a motor. The actuator 78 may be used, for example, for moving the patient’s body part and/or for resisting forces by the patient.

The internal sensors 76 may measure one or more operating characteristics of the treatment apparatus 70 such as, for example, a force, a position, a speed, a velocity, and/or an acceleration. In some embodiments, the internal sensors 76 may include a position sensor configured to measure at least one of a linear motion or an angular motion of a body part of the patient. For example, an internal sensor 76 in the form of a position sensor may measure a distance that the patient is able to move a part of the treatment apparatus 70, where such distance may correspond to a range of motion that the patient’s body part is able to achieve. In some embodiments, the internal sensors 76 may include a force sensor configured to measure a force applied by the patient. For example, an internal sensor 76 in the form of a force sensor may measure a force or weight the patient is able to apply, using a particular body part, to the treatment apparatus 70.

The system 10 shown in FIG. 1 also includes an ambulation sensor 82, which communicates with the server 30 via the local communication interface 68 of the patient interface 50. The ambulation sensor 82 may track and store a number of steps taken by the patient. In some embodiments, the ambulation sensor 82 may take the form of a wristband, wristwatch, or smart watch. In some embodiments, the ambulation sensor 82 may be integrated within a phone, such as a smartphone.

The system 10 shown in FIG. 1 also includes a goniometer 84, which communicates with the server 30 via the local communication interface 68 of the patient interface 50. The goniometer 84 measures an angle of the patient’s body part. For example, the goniometer 84 may measure the angle of flex of a patient’s knee or elbow or shoulder.

The system 10 shown in FIG. 1 also includes a pressure sensor 86, which communicates with the server 30 via the local communication interface 68 of the patient interface 50. The pressure sensor 86 measures an amount of pressure or weight applied by a body part of the patient. For example, pressure sensor 86 may measure an amount of force applied by a patient’s foot when pedaling a stationary bike.

The system 10 shown in FIG. 1 also includes a supervisory interface 90 which may be similar or identical to the clinician interface 20. In some embodiments, the supervisory interface 90 may have enhanced functionality beyond what is provided on the clinician interface 20. The supervisory interface 90 may be configured for use by a person having responsibility for the treatment plan, such as an orthopedic surgeon.

The system 10 shown in FIG. 1 also includes a reporting interface 92 which may be similar or identical to the clinician interface 20. In some embodiments, the reporting interface 92 may have less functionality from what is provided on the clinician interface 20. For example, the reporting interface 92 may not have the ability to modify a treatment plan. Such a reporting interface 92 may be used, for example, by a biller to determine the use of the system 10 for billing purposes. In another example, the reporting interface 92 may not have the ability to display patient identifiable information, presenting only pseudonymized data and/or anonymized data for certain data fields concerning a data subject and/or for certain data fields concerning a quasi-identifier of the data subject. Such a reporting interface 92 may be used, for example, by a researcher to determine various effects of a treatment plan on different patients.

The system 10 includes an assistant interface 94 for an assistant, such as a doctor, a nurse, a physical therapist, or a technician, to remotely communicate with the patient interface 50 and/or the treatment apparatus 70. Such remote communications may enable the assistant to provide assistance or guidance to a patient using the system 10. More specifically, the assistant interface 94 is configured to communicate a telemedicine signal 96, 97, 98a, 98b, 99a, 99b with the patient interface 50 via a network connection such as, for example, via the first network 34 and/or the second network 58. The telemedicine signal 96, 97, 98a, 98b, 99a, 99b comprises one of an audio signal 96, an audiovisual signal 97, an interface control signal 98a for controlling a function of the patient interface 50, an interface monitor signal 98b for monitoring a status of the patient interface 50, an apparatus control signal 99a for changing an operating parameter of the treatment apparatus 70, and/or an apparatus monitor signal 99b for monitoring a status of the treatment apparatus 70. In some embodiments, each of the control signals 98a, 99a may be unidirectional, conveying commands from the assistant interface 94 to the patient interface 50. In some embodiments, in response to successfully receiving a control signal 98a, 99a and/or to communicate successful and/or unsuccessful implementation of the requested control action, an acknowledgement message may be sent from the patient interface 50 to the assistant interface 94. In some embodiments, each of the monitor signals 98b, 99b may be unidirectional, status-information commands from the patient interface 50 to the assistant interface 94. In some embodiments, an acknowledgement message may be sent from the assistant interface 94 to the patient interface 50 in response to successfully receiving one of the monitor signals 98b, 99b.

In some embodiments, the patient interface 50 may be configured as a pass-through for the apparatus control signals 99a and the apparatus monitor signals 99b between the treatment apparatus 70 and one or more other devices, such as the assistant interface 94 and/or the server 30. For example, the patient interface 50 may be configured to transmit an apparatus control signal 99a to the treatment apparatus 70 in response to an apparatus control signal 99a within the telemedicine signal 96, 97, 98a, 98b, 99a, 99b from the assistant interface 94. In some embodiments, the assistant interface 94 transmits the apparatus control signal 99a (e.g., control instruction that causes an operating parameter of the treatment apparatus 70 to change) to the treatment apparatus 70 via any suitable network disclosed herein.

In some embodiments, the assistant interface 94 may be presented on a shared physical device as the clinician interface 20. For example, the clinician interface 20 may include one or more screens that implement the assistant interface 94. Alternatively or additionally, the clinician interface 20 may include additional hardware components, such as a video camera, a speaker, and/or a microphone, to implement aspects of the assistant interface 94.

In some embodiments, one or more portions of the telemedicine signal 96, 97, 98a, 98b, 99a, 99b may be generated from a prerecorded source (e.g., an audio recording, a video recording, or an animation) for presentation by the output device 54 of the patient interface 50. For example, a tutorial video may be streamed from the server 30 and presented upon the patient interface 50. Content from the prerecorded source may be requested by the patient via the patient interface 50. Alternatively, via a control on the assistant interface 94, the assistant may cause content from the prerecorded source to be played on the patient interface 50.

The assistant interface 94 includes an assistant input device 22 and an assistant display 24, which may be collectively called an assistant user interface 22, 24. The assistant input device 22 may include one or more of a telephone, a keyboard, a mouse, a trackpad, or a touch screen, for example. Alternatively or additionally, the assistant input device 22 may include one or more microphones. In some embodiments, the one or more microphones may take the form of a telephone handset, headset, or wide-area microphone or microphones configured for the assistant to speak to a patient via the patient interface 50. In some embodiments, assistant input device 22 may be configured to provide voice-based functionalities, with hardware and/or software configured to interpret spoken instructions by the assistant by using the one or more microphones. The assistant input device 22 may include functionality provided by or similar to existing voice-based assistants such as Siri by Apple, Alexa by Amazon, Google Assistant, or Bixby by Samsung. The assistant input device 22 may include other hardware and/or software components. The assistant input device 22 may include one or more general purpose devices and/or special-purpose devices.

The assistant display 24 may take one or more different forms including, for example, a computer monitor or display screen on a tablet, a smartphone, or a smart watch. The assistant display 24 may include other hardware and/or software components such as projectors, virtual reality capabilities, or augmented reality capabilities, etc. The assistant display 24 may incorporate various different visual, audio, or other presentation technologies. For example, the assistant display 24 may include a non-visual display, such as an audio signal, which may include spoken language and/or other sounds such as tones, chimes, melodies, and/or compositions, which may signal different conditions and/or directions. The assistant display 24 may comprise one or more different display screens presenting various data and/or interfaces or controls for use by the assistant. The assistant display 24 may include graphics, which may be presented by a web-based interface and/or by a computer program or application (App.).

In some embodiments, the system 10 may provide computer translation of language from the assistant interface 94 to the patient interface 50 and/or vice-versa. The computer translation of language may include computer translation of spoken language and/or computer translation of text. Additionally or alternatively, the system 10 may provide voice recognition and/or spoken pronunciation of text. For example, the system 10 may convert spoken words to printed text and/or the system 10 may audibly speak language from printed text. The system 10 may be configured to recognize spoken words by any or all of the patient, the clinician, and/or the healthcare provider. In some embodiments, the system 10 may be configured to recognize and react to spoken requests or commands by the patient. For example, in response to a verbal command by the patient (which may be given in any one of several different languages), the system 10 may automatically initiate a telemedicine session.

In some embodiments, the server 30 may generate aspects of the assistant display 24 for presentation by the assistant interface 94. For example, the server 30 may include a web server configured to generate the display screens for presentation upon the assistant display 24. For example, the artificial intelligence engine 11 may generate recommended treatment plans and/or excluded treatment plans for patients and generate the display screens including those recommended treatment plans and/or external treatment plans for presentation on the assistant display 24 of the assistant interface 94. In some embodiments, the assistant display 24 may be configured to present a virtualized desktop hosted by the server 30. In some embodiments, the server 30 may be configured to communicate with the assistant interface 94 via the first network 34. In some embodiments, the first network 34 may include a local area network (LAN), such as an Ethernet network.

In some embodiments, the first network 34 may include the Internet, and communications between the server 30 and the assistant interface 94 may be secured via privacy enhancing technologies, such as, for example, by using encryption over a virtual private network (VPN). Alternatively or additionally, the server 30 may be configured to communicate with the assistant interface 94 via one or more networks independent of the first network 34 and/or other communication means, such as a direct wired or wireless communication channel. In some embodiments, the patient interface 50 and the treatment apparatus 70 may each operate from a patient location geographically separate from a location of the assistant interface 94. For example, the patient interface 50 and the treatment apparatus 70 may be used as part of an in-home rehabilitation system, which may be aided remotely by using the assistant interface 94 at a centralized location, such as a clinic or a call center.

In some embodiments, the assistant interface 94 may be one of several different terminals (e.g., computing devices) that may be grouped together, for example, in one or more call centers or at one or more clinicians’ offices. In some embodiments, a plurality of assistant interfaces 94 may be distributed geographically. In some embodiments, a person may work as an assistant remotely from any conventional office infrastructure. Such remote work may be performed, for example, where the assistant interface 94 takes the form of a computer and/or telephone. This remote work functionality may allow for work-from-home arrangements that may include part time and/or flexible work hours for an assistant.

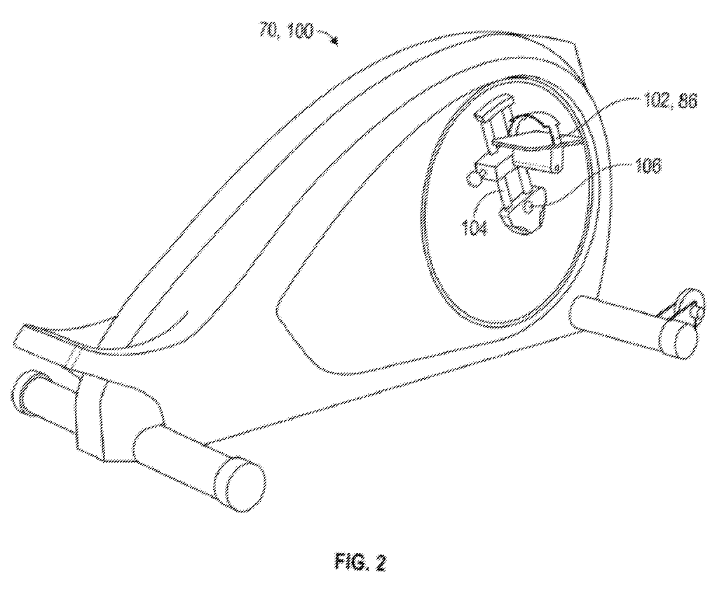

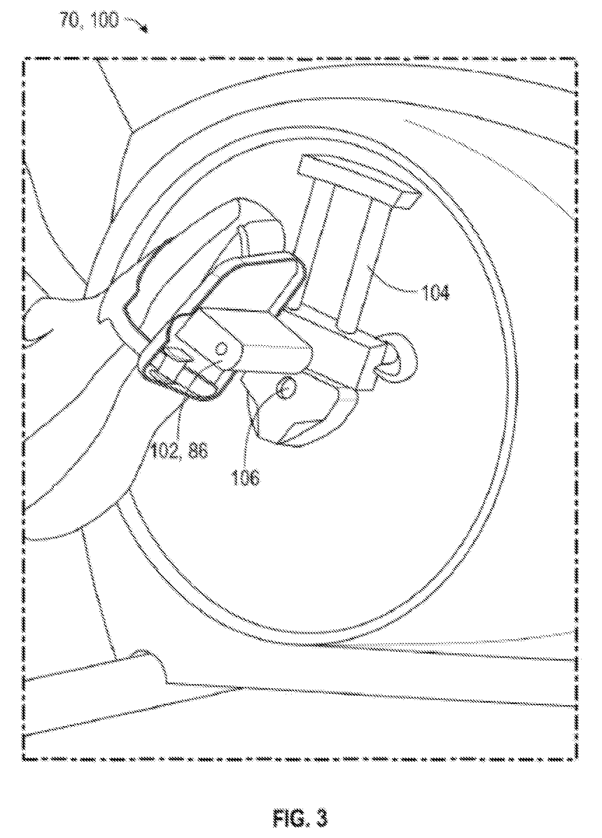

FIGS. 2-3 show an embodiment of a treatment apparatus 70. More specifically, FIG. 2 shows a treatment apparatus 70 in the form of a stationary cycling machine 100, which may be called a stationary bike, for short. The stationary cycling machine 100 includes a set of pedals 102 each attached to a pedal arm 104 for rotation about an axle 106. In some embodiments, and as shown in FIG. 2, the pedals 102 are movable on the pedal arms 104 in order to adjust a range of motion used by the patient in pedaling. For example, the pedals being located inwardly toward the axle 106 corresponds to a smaller range of motion than when the pedals are located outwardly away from the axle 106. A pressure sensor 86 is attached to or embedded within one of the pedals 102 for measuring an amount of force applied by the patient on the pedal 102. The pressure sensor 86 may communicate wirelessly to the treatment apparatus 70 and/or to the patient interface 50.

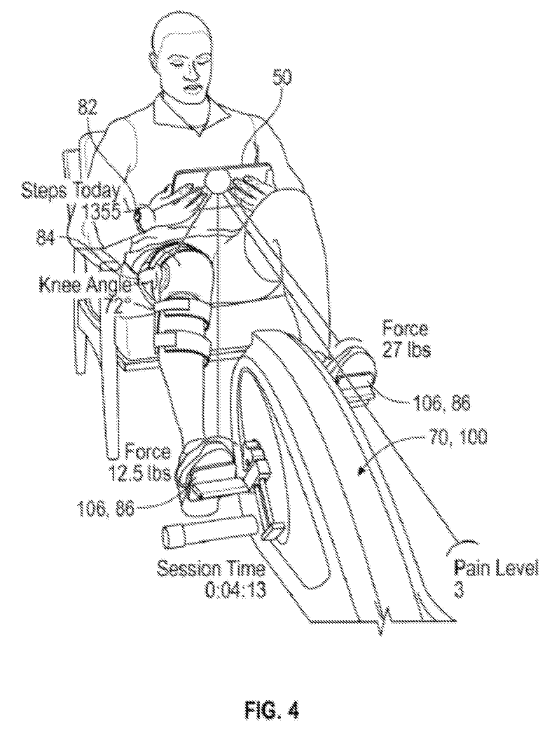

FIG. 4 shows a person (a patient) using the treatment apparatus of FIG. 2, and showing sensors and various data parameters connected to a patient interface 50. The example patient interface 50 is a tablet computer or smartphone, or a phablet, such as an iPad, an iPhone, an Android device, or a Surface tablet, which is held manually by the patient. In some other embodiments, the patient interface 50 may be embedded within or attached to the treatment apparatus 70. FIG. 4 shows the patient wearing the ambulation sensor 82 on his wrist, with a note showing “STEPS TODAY 1355”, indicating that the ambulation sensor 82 has recorded and transmitted that step count to the patient interface 50. FIG. 4 also shows the patient wearing the goniometer 84 on his right knee, with a note showing “KNEE ANGLE 72.degree.”, indicating that the goniometer 84 is measuring and transmitting that knee angle to the patient interface 50. FIG. 4 also shows a right side of one of the pedals 102 with a pressure sensor 86 showing “FORCE 12.5 lbs.,” indicating that the right pedal pressure sensor 86 is measuring and transmitting that force measurement to the patient interface 50. FIG. 4 also shows a left side of one of the pedals 102 with a pressure sensor 86 showing “FORCE 27 lbs.”, indicating that the left pedal pressure sensor 86 is measuring and transmitting that force measurement to the patient interface 50. FIG. 4 also shows other patient data, such as an indicator of “SESSION TIME 0:04:13”, indicating that the patient has been using the treatment apparatus 70 for 4 minutes and 13 seconds. This session time may be determined by the patient interface 50 based on information received from the treatment apparatus 70. FIG. 4 also shows an indicator showing “PAIN LEVEL 3”. Such a pain level may be obtained from the patent in response to a solicitation, such as a question, presented upon the patient interface 50.

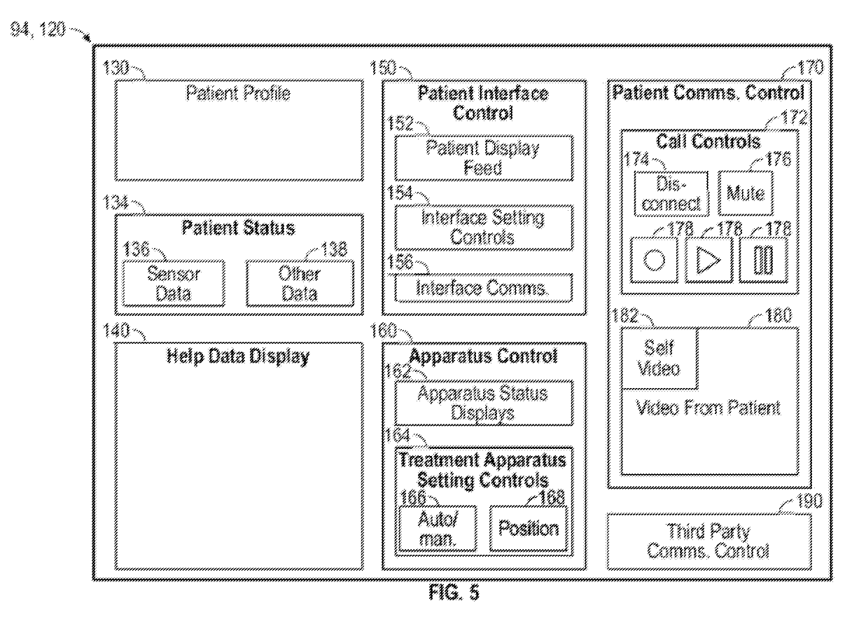

FIG. 5 is an example embodiment of an overview display 120 of the assistant interface 94. Specifically, the overview display 120 presents several different controls and interfaces for the assistant to remotely assist a patient with using the patient interface 50 and/or the treatment apparatus 70. This remote assistance functionality may also be called telemedicine or telehealth.

Specifically, the overview display 120 includes a patient profile display 130 presenting biographical information regarding a patient using the treatment apparatus 70. The patient profile display 130 may take the form of a portion or region of the overview display 120, as shown in FIG. 5, although the patient profile display 130 may take other forms, such as a separate screen or a popup window. In some embodiments, the patient profile display 130 may include a limited subset of the patient’s biographical information. More specifically, the data presented upon the patient profile display 130 may depend upon the assistant’s need for that information. For example, a healthcare provider that is assisting the patient with a medical issue may be provided with medical history information regarding the patient, whereas a technician troubleshooting an issue with the treatment apparatus 70 may be provided with a much more limited set of information regarding the patient. The technician, for example, may be given only the patient’s name. The patient profile display 130 may include pseudonymized data and/or anonymized data or use any privacy enhancing technology to prevent confidential patient data from being communicated in a way that could violate patient confidentiality requirements. Such privacy enhancing technologies may enable compliance with laws, regulations, or other rules of governance such as, but not limited to, the Health Insurance Portability and Accountability Act (HIPAA), or the General Data Protection Regulation (GDPR), wherein the patient may be deemed a “data subject”.

In some embodiments, the patient profile display 130 may present information regarding the treatment plan for the patient to follow in using the treatment apparatus 70. Such treatment plan information may be limited to an assistant who is a healthcare provider, such as a doctor or physical therapist. For example, a healthcare provider assisting the patient with an issue regarding the treatment regimen may be provided with treatment plan information, whereas a technician troubleshooting an issue with the treatment apparatus 70 may not be provided with any information regarding the patient’s treatment plan.

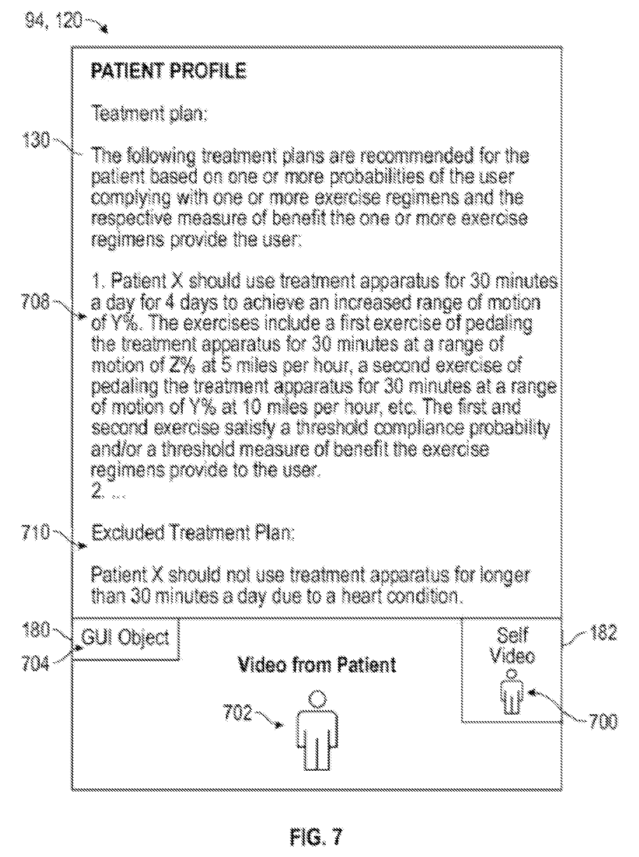

In some embodiments, one or more recommended treatment plans and/or excluded treatment plans may be presented in the patient profile display 130 to the assistant. The one or more recommended treatment plans and/or excluded treatment plans may be generated by the artificial intelligence engine 11 of the server 30 and received from the server 30 in real-time during, inter alia, a telemedicine or telehealth session. An example of presenting the one or more recommended treatment plans and/or excluded treatment plans is described below with reference to FIG. 7.

The example overview display 120 shown in FIG. 5 also includes a patient status display 134 presenting status information regarding a patient using the treatment apparatus. The patient status display 134 may take the form of a portion or region of the overview display 120, as shown in FIG. 5, although the patient status display 134 may take other forms, such as a separate screen or a popup window. The patient status display 134 includes sensor data 136 from one or more of the external sensors 82, 84, 86, and/or from one or more internal sensors 76 of the treatment apparatus 70. In some embodiments, the patient status display 134 may include sensor data from one or more sensors of one or more wearable devices worn by the patient while using the treatment device 70. The one or more wearable devices may include a watch, a bracelet, a necklace, a chest strap, and the like. The one or more wearable devices may be configured to monitor a heartrate, a temperature, a blood pressure, one or more vital signs, and the like of the patient while the patient is using the treatment device 70. In some embodiments, the patient status display 134 may present other data 138 regarding the patient, such as last reported pain level, or progress within a treatment plan.

User access controls may be used to limit access, including what data is available to be viewed and/or modified, on any or all of the user interfaces 20, 50, 90, 92, 94 of the system 10. In some embodiments, user access controls may be employed to control what information is available to any given person using the system 10. For example, data presented on the assistant interface 94 may be controlled by user access controls, with permissions set depending on the assistant/user’s need for and/or qualifications to view that information.

The example overview display 120 shown in FIG. 5 also includes a help data display 140 presenting information for the assistant to use in assisting the patient. The help data display 140 may take the form of a portion or region of the overview display 120, as shown in FIG. 5. The help data display 140 may take other forms, such as a separate screen or a popup window. The help data display 140 may include, for example, presenting answers to frequently asked questions regarding use of the patient interface 50 and/or the treatment apparatus 70. The help data display 140 may also include research data or best practices. In some embodiments, the help data display 140 may present scripts for answers or explanations in response to patient questions. In some embodiments, the help data display 140 may present flow charts or walk-throughs for the assistant to use in determining a root cause and/or solution to a patient’s problem. In some embodiments, the assistant interface 94 may present two or more help data displays 140, which may be the same or different, for simultaneous presentation of help data for use by the assistant. for example, a first help data display may be used to present a troubleshooting flowchart to determine the source of a patient’s problem, and a second help data display may present script information for the assistant to read to the patient, such information to preferably include directions for the patient to perform some action, which may help to narrow down or solve the problem. In some embodiments, based upon inputs to the troubleshooting flowchart in the first help data display, the second help data display may automatically populate with script information.

The example overview display 120 shown in FIG. 5 also includes a patient interface control 150 presenting information regarding the patient interface 50, and/or to modify one or more settings of the patient interface 50. The patient interface control 150 may take the form of a portion or region of the overview display 120, as shown in FIG. 5. The patient interface control 150 may take other forms, such as a separate screen or a popup window. The patient interface control 150 may present information communicated to the assistant interface 94 via one or more of the interface monitor signals 98b. As shown in FIG. 5, the patient interface control 150 includes a display feed 152 of the display presented by the patient interface 50. In some embodiments, the display feed 152 may include a live copy of the display screen currently being presented to the patient by the patient interface 50. In other words, the display feed 152 may present an image of what is presented on a display screen of the patient interface 50. In some embodiments, the display feed 152 may include abbreviated information regarding the display screen currently being presented by the patient interface 50, such as a screen name or a screen number. The patient interface control 150 may include a patient interface setting control 154 for the assistant to adjust or to control one or more settings or aspects of the patient interface 50. In some embodiments, the patient interface setting control 154 may cause the assistant interface 94 to generate and/or to transmit an interface control signal 98 for controlling a function or a setting of the patient interface 50.

In some embodiments, the patient interface setting control 154 may include collaborative browsing or co-browsing capability for the assistant to remotely view and/or control the patient interface 50. For example, the patient interface setting control 154 may enable the assistant to remotely enter text to one or more text entry fields on the patient interface 50 and/or to remotely control a cursor on the patient interface 50 using a mouse or touchscreen of the assistant interface 94.

In some embodiments, using the patient interface 50, the patient interface setting control 154 may allow the assistant to change a setting that cannot be changed by the patient. For example, the patient interface 50 may be precluded from accessing a language setting to prevent a patient from inadvertently switching, on the patient interface 50, the language used for the displays, whereas the patient interface setting control 154 may enable the assistant to change the language setting of the patient interface 50. In another example, the patient interface 50 may not be able to change a font size setting to a smaller size in order to prevent a patient from inadvertently switching the font size used for the displays on the patient interface 50 such that the display would become illegible to the patient, whereas the patient interface setting control 154 may provide for the assistant to change the font size setting of the patient interface 50.

The example overview display 120 shown in FIG. 5 also includes an interface communications display 156 showing the status of communications between the patient interface 50 and one or more other devices 70, 82, 84, such as the treatment apparatus 70, the ambulation sensor 82, and/or the goniometer 84. The interface communications display 156 may take the form of a portion or region of the overview display 120, as shown in FIG. 5. The interface communications display 156 may take other forms, such as a separate screen or a popup window. The interface communications display 156 may include controls for the assistant to remotely modify communications with one or more of the other devices 70, 82, 84. For example, the assistant may remotely command the patient interface 50 to reset communications with one of the other devices 70, 82, 84, or to establish communications with a new one of the other devices 70, 82, 84. This functionality may be used, for example, where the patient has a problem with one of the other devices 70, 82, 84, or where the patient receives a new or a replacement one of the other devices 70, 82, 84.

The example overview display 120 shown in FIG. 5 also includes an apparatus control 160 for the assistant to view and/or to control information regarding the treatment apparatus 70. The apparatus control 160 may take the form of a portion or region of the overview display 120, as shown in FIG. 5. The apparatus control 160 may take other forms, such as a separate screen or a popup window. The apparatus control 160 may include an apparatus status display 162 with information regarding the current status of the apparatus. The apparatus status display 162 may present information communicated to the assistant interface 94 via one or more of the apparatus monitor signals 99b. The apparatus status display 162 may indicate whether the treatment apparatus 70 is currently communicating with the patient interface 50. The apparatus status display 162 may present other current and/or historical information regarding the status of the treatment apparatus 70.

The apparatus control 160 may include an apparatus setting control 164 for the assistant to adjust or control one or more aspects of the treatment apparatus 70. The apparatus setting control 164 may cause the assistant interface 94 to generate and/or to transmit an apparatus control signal 99a for changing an operating parameter of the treatment apparatus 70, (e.g., a pedal radius setting, a resistance setting, a target RPM, other suitable characteristics of the treatment device 70, or a combination thereof).

The apparatus setting control 164 may include a mode button 166 and a position control 168, which may be used in conjunction for the assistant to place an actuator 78 of the treatment apparatus 70 in a manual mode, after which a setting, such as a position or a speed of the actuator 78, can be changed using the position control 168. The mode button 166 may provide for a setting, such as a position, to be toggled between automatic and manual modes. In some embodiments, one or more settings may be adjustable at any time, and without having an associated auto/manual mode. In some embodiments, the assistant may change an operating parameter of the treatment apparatus 70, such as a pedal radius setting, while the patient is actively using the treatment apparatus 70. Such “on the fly” adjustment may or may not be available to the patient using the patient interface 50. In some embodiments, the apparatus setting control 164 may allow the assistant to change a setting that cannot be changed by the patient using the patient interface 50. For example, the patient interface 50 may be precluded from changing a preconfigured setting, such as a height or a tilt setting of the treatment apparatus 70, whereas the apparatus setting control 164 may provide for the assistant to change the height or tilt setting of the treatment apparatus 70.

The example overview display 120 shown in FIG. 5 also includes a patient communications control 170 for controlling an audio or an audiovisual communications session with the patient interface 50. The communications session with the patient interface 50 may comprise a live feed from the assistant interface 94 for presentation by the output device of the patient interface 50. The live feed may take the form of an audio feed and/or a video feed. In some embodiments, the patient interface 50 may be configured to provide two-way audio or audiovisual communications with a person using the assistant interface 94. Specifically, the communications session with the patient interface 50 may include bidirectional (two-way) video or audiovisual feeds, with each of the patient interface 50 and the assistant interface 94 presenting video of the other one. In some embodiments, the patient interface 50 may present video from the assistant interface 94, while the assistant interface 94 presents only audio or the assistant interface 94 presents no live audio or visual signal from the patient interface 50. In some embodiments, the assistant interface 94 may present video from the patient interface 50, while the patient interface 50 presents only audio or the patient interface 50 presents no live audio or visual signal from the assistant interface 94.

In some embodiments, the audio or an audiovisual communications session with the patient interface 50 may take place, at least in part, while the patient is performing the rehabilitation regimen upon the body part. The patient communications control 170 may take the form of a portion or region of the overview display 120, as shown in FIG. 5. The patient communications control 170 may take other forms, such as a separate screen or a popup window. The audio and/or audiovisual communications may be processed and/or directed by the assistant interface 94 and/or by another device or devices, such as a telephone system, or a videoconferencing system used by the assistant while the assistant uses the assistant interface 94. Alternatively or additionally, the audio and/or audiovisual communications may include communications with a third party. For example, the system 10 may enable the assistant to initiate a 3-way conversation regarding use of a particular piece of hardware or software, with the patient and a subject matter expert, such as a medical professional or a specialist. The example patient communications control 170 shown in FIG. 5 includes call controls 172 for the assistant to use in managing various aspects of the audio or audiovisual communications with the patient. The call controls 172 include a disconnect button 174 for the assistant to end the audio or audiovisual communications session. The call controls 172 also include a mute button 176 to temporarily silence an audio or audiovisual signal from the assistant interface 94. In some embodiments, the call controls 172 may include other features, such as a hold button (not shown). The call controls 172 also include one or more record/playback controls 178, such as record, play, and pause buttons to control, with the patient interface 50, recording and/or playback of audio and/or video from the teleconference session (e.g., which may be referred to herein as the virtual conference room). The call controls 172 also include a video feed display 180 for presenting still and/or video images from the patient interface 50, and a self-video display 182 showing the current image of the assistant using the assistant interface. The self-video display 182 may be presented as a picture-in-picture format, within a section of the video feed display 180, as shown in FIG. 5. Alternatively or additionally, the self-video display 182 may be presented separately and/or independently from the video feed display 180.

The example overview display 120 shown in FIG. 5 also includes a third party communications control 190 for use in conducting audio and/or audiovisual communications with a third party. The third party communications control 190 may take the form of a portion or region of the overview display 120, as shown in FIG. 5. The third party communications control 190 may take other forms, such as a display on a separate screen or a popup window. The third party communications control 190 may include one or more controls, such as a contact list and/or buttons or controls to contact a third party regarding use of a particular piece of hardware or software, e.g., a subject matter expert, such as a medical professional or a specialist. The third party communications control 190 may include conference calling capability for the third party to simultaneously communicate with both the assistant via the assistant interface 94, and with the patient via the patient interface 50. For example, the system 10 may provide for the assistant to initiate a 3-way conversation with the patient and the third party.

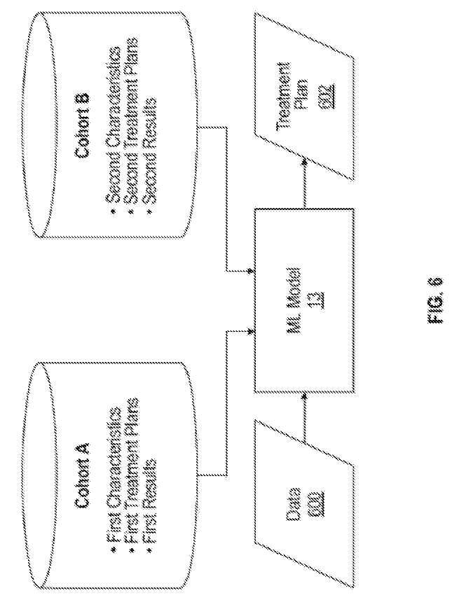

FIG. 6 shows an example block diagram of training a machine learning model 13 to output, based on data 600 pertaining to the patient, a treatment plan 602 for the patient according to the present disclosure. Data pertaining to other patients may be received by the server 30. The other patients may have used various treatment apparatuses to perform treatment plans. The data may include characteristics of the other patients, the details of the treatment plans performed by the other patients, and/or the results of performing the treatment plans (e.g., a percent of recovery of a portion of the patients’ bodies, an amount of recovery of a portion of the patients’ bodies, an amount of increase or decrease in muscle strength of a portion of patients’ bodies, an amount of increase or decrease in range of motion of a portion of patients’ bodies, etc.).

As depicted, the data has been assigned to different cohorts. Cohort A includes data for patients having similar first characteristics, first treatment plans, and first results. Cohort B includes data for patients having similar second characteristics, second treatment plans, and second results. For example, cohort A may include first characteristics of patients in their twenties without any medical conditions who underwent surgery for a broken limb; their treatment plans may include a certain treatment protocol (e.g., use the treatment apparatus 70 for 30 minutes 5 times a week for 3 weeks, wherein values for the properties, configurations, and/or settings of the treatment apparatus 70 are set to X (where X is a numerical value) for the first two weeks and to Y (where Y is a numerical value) for the last week).

Cohort A and cohort B may be included in a training dataset used to train the machine learning model 13. The machine learning model 13 may be trained to match a pattern between characteristics for each cohort and output the treatment plan that provides the result. Accordingly, when the data 600 for a new patient is input into the trained machine learning model 13, the trained machine learning model 13 may match the characteristics included in the data 600 with characteristics in either cohort A or cohort B and output the appropriate treatment plan 602. In some embodiments, the machine learning model 13 may be trained to output one or more excluded treatment plans that should not be performed by the new patient.

FIG. 7 shows an embodiment of an overview display 120 of the assistant interface 94 presenting recommended treatment plans and excluded treatment plans in real-time during a telemedicine session according to the present disclosure. As depicted, the overview display 120 only includes sections for the patient profile 130 and the video feed display 180, including the self-video display 182. Any suitable configuration of controls and interfaces of the overview display 120 described with reference to FIG. 5 may be presented in addition to or instead of the patient profile 130, the video feed display 180, and the self-video display 182.

The healthcare provider using the assistant interface 94 (e.g., computing device) during the telemedicine session may be presented in the self-video 182 in a portion of the overview display 120 (e.g., user interface presented on a display screen 24 of the assistant interface 94) that also presents a video from the patient in the video feed display 180. Further, the video feed display 180 may also include a graphical user interface (GUI) object 700 (e.g., a button) that enables the healthcare provider to share on the patient interface 50, in real-time or near real-time during the telemedicine session, the recommended treatment plans and/or the excluded treatment plans with the patient. The healthcare provider may select the GUI object 700 to share the recommended treatment plans and/or the excluded treatment plans. As depicted, another portion of the overview display 120 includes the patient profile display 130.

In FIG. 7, the patient profile display 130 is presenting two example recommended treatment plans 708 and one example excluded treatment plan 710. As described herein, the treatment plans may be recommended based on the one or more probabilities and the respective measure of benefit the one or more exercises provide the user. The trained machine learning models 13 may (i) use treatment data pertaining to a user to determine a respective measure of benefit which one or more exercise regimens provide the user, (ii) determine one or more probabilities of the user associated with complying with the one or more exercise regimens, and (iii) generate, using the one or more probabilities and the respective measure of benefit the one or more exercises provide to the user, the treatment plan. In some embodiments, the one or more trained machine learning models 13 may generate treatment plans including exercises associated with a certain threshold (e.g., any suitable percentage metric, value, percentage, number, indicator, probability, etc., which may be configurable) associated with the user complying with the one or more exercise regimens to enable achieving a higher user compliance with the treatment plan. In some embodiments, the one or more trained machine learning models 13 may generate treatment plans including exercises associated with a certain threshold (e.g., any suitable percentage metric, value, percentage, number, indicator, probability, etc., which may be configurable) associated with one or more measures of benefit the exercises provide to the user to enable achieving the benefits (e.g., strength, flexibility, range of motion, etc.) at a faster rate, at a greater proportion, etc. In some embodiments, when both the measures of benefit and the probability of compliance are considered by the trained machine learning models 13, each of the measures of benefit and the probability of compliance may be associated with a different weight, such different weight causing one to be more influential than the other. Such techniques may enable configuring which parameter (e.g., probability of compliance or measures of benefit) is more desirable to consider more heavily during generation of the treatment plan.

For example, as depicted, the patient profile display 130 presents “The following treatment plans are recommended for the patient based on one or more probabilities of the user complying with one or more exercise regimens and the respective measure of benefit the one or more exercises provide the user.” Then, the patient profile display 130 presents a first recommended treatment plan.

As depicted, treatment plan “1” indicates “Patient X should use treatment apparatus for 30 minutes a day for 4 days to achieve an increased range of motion of Y %. The exercises include a first exercise of pedaling the treatment apparatus for 30 minutes at a range of motion of Z % at 5 miles per hour, a second exercise of pedaling the treatment apparatus for 30 minutes at a range of motion of Y % at 10 miles per hour, etc. The first and second exercise satisfy a threshold compliance probability and/or a threshold measure of benefit which the exercise regimens provide to the user.” Accordingly, the treatment plan generated includes a first and second exercise, etc. that increase the range of motion of Y %. Further, in some embodiments, the exercises are indicated as satisfying a threshold compliance probability and/or a threshold measure of benefit which the exercise regimens provide to the user. Each of the exercises may specify any suitable parameter of the exercise and/or treatment apparatus 70 (e.g., duration of exercise, speed of motor of the treatment apparatus 70, range of motion setting of pedals, etc.). This specific example and all such examples elsewhere herein are not intended to limit in any way the generated treatment plan from recommending any suitable number and/or type of exercise.

Recommended treatment plan “2” may specify, based on a desired benefit, an indication of a probability of compliance, or some combination thereof, and different exercises for the user to perform.

As depicted, the patient profile display 130 may also present the excluded treatment plans 710. These types of treatment plans are shown to the assistant using the assistant interface 94 to alert the assistant not to recommend certain portions of a treatment plan to the patient. For example, the excluded treatment plan could specify the following: “Patient X should not use treatment apparatus for longer than 30 minutes a day due to a heart condition.” Specifically, the excluded treatment plan points out a limitation of a treatment protocol where, due to a heart condition, Patient X should not exercise for more than 30 minutes a day. The excluded treatment plans may be based on treatment data (e.g., characteristics of the user, characteristics of the treatment apparatus 70, or the like).

The assistant may select the treatment plan for the patient on the overview display 120. For example, the assistant may use an input peripheral (e.g., mouse, touchscreen, microphone, keyboard, etc.) to select from the treatment plans 708 for the patient.

In any event, the assistant may select the treatment plan for the patient to follow to achieve a desired result. The selected treatment plan may be transmitted to the patient interface 50 for presentation. The patient may view the selected treatment plan on the patient interface 50. In some embodiments, the assistant and the patient may discuss during the telemedicine session the details (e.g., treatment protocol using treatment apparatus 70, diet regimen, medication regimen, etc.) in real-time or in near real-time. In some embodiments, as discussed further with reference to method 1000 of FIG. 10 below, the server 30 may control, based on the selected treatment plan and during the telemedicine session, the treatment apparatus 70 as the user uses the treatment apparatus 70.

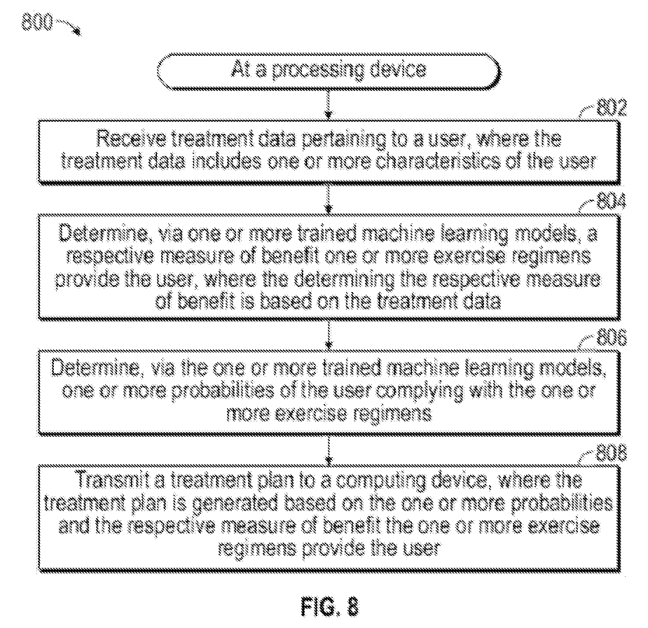

FIG. 8 shows an example embodiment of a method 800 for optimizing a treatment plan for a user to increase a probability of the user complying with the treatment plan according to the present disclosure. The method 800 is performed by processing logic that may include hardware (circuitry, dedicated logic, etc.), software (such as is run on a general-purpose computer system or a dedicated machine), or a combination of both. The method 800 and/or each of its individual functions, routines, other methods, scripts, subroutines, or operations may be performed by one or more processors of a computing device (e.g., any component of FIG. 1, such as server 30 executing the artificial intelligence engine 11). In certain implementations, the method 800 may be performed by a single processing thread. Alternatively, the method 800 may be performed by two or more processing threads, each thread implementing one or more individual functions or routines; or other methods, scripts, subroutines, or operations of the methods.

For simplicity of explanation, the method 800 is depicted and described as a series of operations. However, operations in accordance with this disclosure can occur in various orders and/or concurrently, and/or with other operations not presented and described herein. For example, the operations depicted in the method 800 may occur in combination with any other operation of any other method disclosed herein. Furthermore, not all illustrated operations may be required to implement the method 800 in accordance with the disclosed subject matter. In addition, those skilled in the art will understand and appreciate that the method 800 could alternatively be represented as a series of interrelated states via a state diagram, a directed graph, a deterministic finite state automaton, a non-deterministic finite state automaton, a Markov diagram, or event diagrams.

At 802, the processing device may receive treatment data pertaining to a user (e.g., patient, volunteer, trainee, assistant, healthcare provider, instructor, etc.). The treatment data may include one or more characteristics (e.g., vital-sign or other measurements; performance; demographic; psychographic; geographic; diagnostic; measurement- or test-based; medically historic; etiologic; cohort-associative; differentially diagnostic; surgical, physically therapeutic, pharmacologic and other treatment(s) recommended; arterial blood gas and/or oxygenation levels or percentages; psychographics; etc.) of the user. The treatment data may include one or more characteristics of the treatment apparatus 70. In some embodiments, the one or more characteristics of the treatment apparatus 70 may include a make (e.g., identity of entity that designed, manufactured, etc. the treatment apparatus 70) of the treatment apparatus 70, a model (e.g., model number or other identifier of the model) of the treatment apparatus 70, a year (e.g., year of manufacturing) of the treatment apparatus 70, operational parameters (e.g., motor temperature during operation; status of each sensor included in or associated with the treatment apparatus 70; the patient, or the environment; vibration measurements of the treatment apparatus 70 in operation; measurements of static and/or dynamic forces exerted on the treatment apparatus 70; etc.) of the treatment apparatus 70, settings (e.g., range of motion setting; speed setting; required pedal force setting; etc.) of the treatment apparatus 70, and the like. In some embodiments, the characteristics of the user and/or the characteristics of the treatment apparatus 70 may be tracked over time to obtain historical data pertaining to the characteristics of the user and/or the treatment apparatus 70. The foregoing embodiments shall also be deemed to include the use of any optional internal components or of any external components attachable to, but separate from the treatment apparatus itself. “Attachable” as used herein shall be physically, electronically, mechanically, virtually or in an augmented reality manner.

In some embodiments, when generating a treatment plan, the characteristics of the user and/or treatment apparatus 70 may be used. For example, certain exercises may be selected or excluded based on the characteristics of the user and/or treatment apparatus 70. For example, if the user has a heart condition, high intensity exercises may be excluded in a treatment plan. In another example, a characteristic of the treatment apparatus 70 may indicate the motor shudders, stalls or otherwise runs improperly at a certain number of revolutions per minute. In order to extend the lifetime of the treatment apparatus 70, the treatment plan may exclude exercises that include operating the motor at that certain revolutions per minute or at a prescribed manufacturing tolerance within those certain revolutions per minute.

At 804, the processing device may determine, via one or more trained machine learning models 13, a respective measure of benefit with which one or more exercises provide the user. In some embodiments, based on the treatment data, the processing device may execute the one or more trained machine learning models 13 to determine the respective measures of benefit. For example, the treatment data may include the characteristics of the user (e.g., heartrate, vital-sign, medical condition, injury, surgery, etc.), and the one or more trained machine learning models may receive the treatment data and output the respective measure of benefit with which one or more exercises provide the user. For example, if the user has a heart condition, a high intensity exercise may provide a negative benefit to the user, and thus, the trained machine learning model may output a negative measure of benefit for the high intensity exercise for the user. In another example, an exercise including pedaling at a certain range of motion may have a positive benefit for a user recovering from a certain surgery, and thus, the trained machine learning model may output a positive measure of benefit for the exercise regimen for the user.

At 806, the processing device may determine, via the one or more trained machine learning models 13, one or more probabilities associated with the user complying with the one or more exercise regimens. In some embodiments, the relationship between the one or more probabilities associated with the user complying with the one or more exercise regimens may be one to one, one to many, many to one, or many to many. The one or more probabilities of compliance may refer to a metric (e.g., value, percentage, number, indicator, probability, etc.) associated with a probability the user will comply with an exercise regimen. In some embodiments, the processing device may execute the one or more trained machine learning models 13 to determine the one or more probabilities based on (i) historical data pertaining to the user, another user, or both, (ii) received feedback from the user, another user, or both, (iii) received feedback from a treatment apparatus used by the user, or (iv) some combination thereof.

For example, historical data pertaining to the user may indicate a history of the user previously performing one or more of the exercises. In some instances, at a first time, the user may perform a first exercise to completion. At a second time, the user may terminate a second exercise prior to completion. Feedback data from the user and/or the treatment apparatus 70 may be obtained before, during, and after each exercise performed by the user. The trained machine learning model may use any combination of data (e.g., (i) historical data pertaining to the user, another user, or both, (ii) received feedback from the user, another user, or both, (iii) received feedback from a treatment apparatus used by the user) described above to learn a user compliance profile for each of the one or more exercises. The term “user compliance profile” may refer to a collection of histories of the user complying with the one or more exercise regimens. In some embodiments, the trained machine learning model may use the user compliance profile, among other data (e.g., characteristics of the treatment apparatus 70), to determine the one or more probabilities of the user complying with the one or more exercise regimens.

At 808, the processing device may transmit a treatment plan to a computing device. The computing device may be any suitable interface described herein. For example, the treatment plan may be transmitted to the assistant interface 94 for presentation to a healthcare provider, and/or to the patient interface 50 for presentation to the patient. The treatment plan may be generated based on the one or more probabilities and the respective measure of benefit the one or more exercises may provide to the user. In some embodiments, as described further below with reference to the method 1000 of FIG. 10, while the user uses the treatment apparatus 70, the processing device may control, based on the treatment plan, the treatment apparatus 70.

In some embodiments, the processing device may generate, using at least a subset of the one or more exercises, the treatment plan for the user to perform, wherein such performance uses the treatment apparatus 70. The processing device may execute the one or more trained machine learning models 13 to generate the treatment plan based on the respective measure of the benefit the one or more exercises provide to the user, the one or more probabilities associated with the user complying with each of the one or more exercise regimens, or some combination thereof. For example, the one or more trained machine learning models 13 may receive the respective measure of the benefit the one or more exercises provide to the user, the one or more probabilities of the user associated with complying with each of the one or more exercise regimens, or some combination thereof as input and output the treatment plan.

In some embodiments, during generation of the treatment plan, the processing device may more heavily or less heavily weight the probability of the user complying than the respective measure of benefit the one or more exercise regimens provide to the user. During generation of the treatment plan, such a technique may enable one of the factors (e.g., the probability of the user complying or the respective measure of benefit the one or more exercise regimens provide to the user) to become more important than the other factor. For example, if desirable to select exercises that the user is more likely to comply with in a treatment plan, then the one or more probabilities of the user associated with complying with each of the one or more exercise regimens may receive a higher weight than one or more measures of exercise benefit factors. In another example, if desirable to obtain certain benefits provided by exercises, then the measure of benefit an exercise regimen provides to a user may receive a higher weight than the user compliance probability factor. The weight may be any suitable value, number, modifier, percentage, probability, etc.

In some embodiments, the processing device may generate the treatment plan using a non-parametric model, a parametric model, or a combination of both a non-parametric model and a parametric model. In statistics, a parametric model or finite-dimensional model refers to probability distributions that have a finite number of parameters. Non-parametric models include model structures not specified a priori but instead determined from data. In some embodiments, the processing device may generate the treatment plan using a probability density function, a Bayesian prediction model, a Markovian prediction model, or any other suitable mathematically-based prediction model. A Bayesian prediction model is used in statistical inference where Bayes’ theorem is used to update the probability for a hypothesis as more evidence or information becomes available. Bayes’ theorem may describe the probability of an event, based on prior knowledge of conditions that might be related to the event. For example, as additional data (e.g., user compliance data for certain exercises, characteristics of users, characteristics of treatment apparatuses, and the like) are obtained, the probabilities of compliance for users for performing exercise regimens may be continuously updated. The trained machine learning models 13 may use the Bayesian prediction model and, in preferred embodiments, continuously, constantly or frequently be re-trained with additional data obtained by the artificial intelligence engine 11 to update the probabilities of compliance, and/or the respective measure of benefit one or more exercises may provide to a user.

In some embodiments, the processing device may generate the treatment plan based on a set of factors. In some embodiments, the set of factors may include an amount, quality or other quality of sleep associated with the user, information pertaining to a diet of the user, information pertaining to an eating schedule of the user, information pertaining to an age of the user, information pertaining to a sex of the user, information pertaining to a gender of the user, an indication of a mental state of the user, information pertaining to a genetic condition of the user, information pertaining to a disease state of the user, an indication of an energy level of the user, or some combination thereof. For example, the set of factors may be included in the training data used to train and/or re-train the one or more machine learning models 13. For example, the set of factors may be labeled as corresponding to treatment data indicative of certain measures of benefit one or more exercises provide to the user, probabilities of the user complying with the one or more exercise regimens, or both.

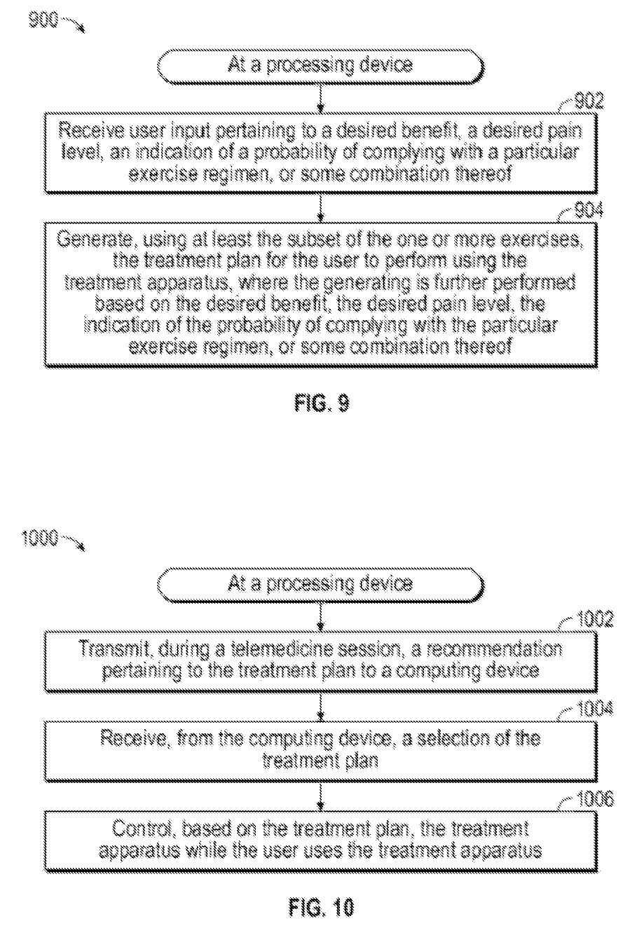

FIG. 9 shows an example embodiment of a method 900 for generating a treatment plan based on a desired benefit, a desired pain level, an indication of a probability associated with complying with the particular exercise regimen, or some combination thereof, according to some embodiments. Method 900 includes operations performed by processors of a computing device (e.g., any component of FIG. 1, such as server 30 executing the artificial intelligence engine 11). In some embodiments, one or more operations of the method 900 are implemented in computer instructions stored on a memory device and executed by a processing device. The method 900 may be performed in the same or a similar manner as described above in regard to method 800. The operations of the method 900 may be performed in some combination with any of the operations of any of the methods described herein.

At 902, the processing device may receive user input pertaining to a desired benefit, a desired pain level, an indication of a probability associated with complying with a particular exercise regimen, or some combination thereof. The user input may be received from the patient interface 50. That is, in some embodiments, the patient interface 50 may present a display including various graphical elements that enable the user to enter a desired benefit of performing an exercise, a desired pain level (e.g., on a scale ranging from 1-10, 1 being the lowest pain level and 10 being the highest pain level), an indication of a probability associated with complying with the particular exercise regimen, or some combination thereof. For example, the user may indicate he or she would not comply with certain exercises (e.g., one-arm push-ups) included in an exercise regimen due to a lack of ability to perform the exercise and/or a lack of desire to perform the exercise. The patient interface 50 may transmit the user input to the processing device (e.g., of the server 30, assistant interface 94, or any suitable interface described herein).

At 904, the processing device may generate, using at least a subset of the one or more exercises, the treatment plan for the user to perform wherein the performance uses the treatment apparatus 70. The processing device may generate the treatment plan based on the user input including the desired benefit, the desired pain level, the indication of the probability associated with complying with the particular exercise regimen, or some combination thereof. For example, if the user selected a desired benefit of improved range of motion of flexion and extension of their knee, then the one or more trained machine learning models 13 may identify, based on treatment data pertaining to the user, exercises that provide the desired benefit. Those identified exercises may be further filtered based on the probabilities of user compliance with the exercise regimens. Accordingly, the one or more machine learning models 13 may be interconnected, such that the output of one or more trained machine learning models that perform function(s) (e.g., determine measures of benefit exercises provide to user) may be provided as input to one or more other trained machine learning models that perform other functions(s) (e.g., determine probabilities of the user complying with the one or more exercise regimens, generate the treatment plan based on the measures of benefit and/or the probabilities of the user complying, etc.).

FIG. 10 shows an example embodiment of a method 1000 for controlling, based on a treatment plan, a treatment apparatus 70 while a user uses the treatment apparatus 70, according to some embodiments. Method 1000 includes operations performed by processors of a computing device (e.g., any component of FIG. 1, such as server 30 executing the artificial intelligence engine 11). In some embodiments, one or more operations of the method 1000 are implemented in computer instructions stored on a memory device and executed by a processing device. The method 1000 may be performed in the same or a similar manner as described above in regard to method 800. The operations of the method 1000 may be performed in some combination with any of the operations of any of the methods described herein.

At 1002, the processing device may transmit, during a telemedicine or telehealth session, a recommendation pertaining to a treatment plan to a computing device (e.g., patient interface 50, assistant interface 94, or any suitable interface described herein). The recommendation may be presented on a display screen of the computing device in real-time (e.g., less than 2 seconds) in a portion of the display screen while another portion of the display screen presents video of a user (e.g., patient, healthcare provider, or any suitable user). The recommendation may also be presented on a display screen of the computing device in near time (e.g., preferably more than or equal to 2 seconds and less than or equal to 10 seconds) or with a suitable time delay necessary for the user of the display screen to be able to observe the display screen.

At 1004, the processing device may receive, from the computing device, a selection of the treatment plan. The user (e.g., patient, healthcare provider, assistant, etc.) may use any suitable input peripheral (e.g., mouse, keyboard, microphone, touchpad, etc.) to select the recommended treatment plan. The computing device may transmit the selection to the processing device of the server 30, which is configured to receive the selection. There may any suitable number of treatment plans presented on the display screen. Each of the treatment plans recommended may provide different results and the healthcare provider may consult, during the telemedicine session, with the user, to discuss which result the user desires. In some embodiments, the recommended treatment plans may only be presented on the computing device of the healthcare provider and not on the computing device of the user (patient interface 50). In some embodiments, the healthcare provider may choose an option presented on the assistant interface 94. The option may cause the treatment plans to be transmitted to the patient interface 50 for presentation. In this way, during the telemedicine session, the healthcare provider and the user may view the treatment plans at the same time in real-time or in near real-time, which may provide for an enhanced user experience for the patient and/or healthcare provider using the computing device.

After the selection of the treatment plan is received at the server 30, at 1006, while the user uses the treatment apparatus 70, the processing device may control, based on the selected treatment plan, the treatment apparatus 70. In some embodiments, controlling the treatment apparatus 70 may include the server 30 generating and transmitting control instructions to the treatment apparatus 70. In some embodiments, controlling the treatment apparatus 70 may include the server 30 generating and transmitting control instructions to the patient interface 50, and the patient interface 50 may transmit the control instructions to the treatment apparatus 70. The control instructions may cause an operating parameter (e.g., speed, orientation, required force, range of motion of pedals, etc.) to be dynamically changed according to the treatment plan (e.g., a range of motion may be changed to a certain setting based on the user achieving a certain range of motion for a certain period of time). The operating parameter may be dynamically changed while the patient uses the treatment apparatus 70 to perform an exercise. In some embodiments, during a telemedicine session between the patient interface 50 and the assistant interface 94, the operating parameter may be dynamically changed in real-time or near real-time.

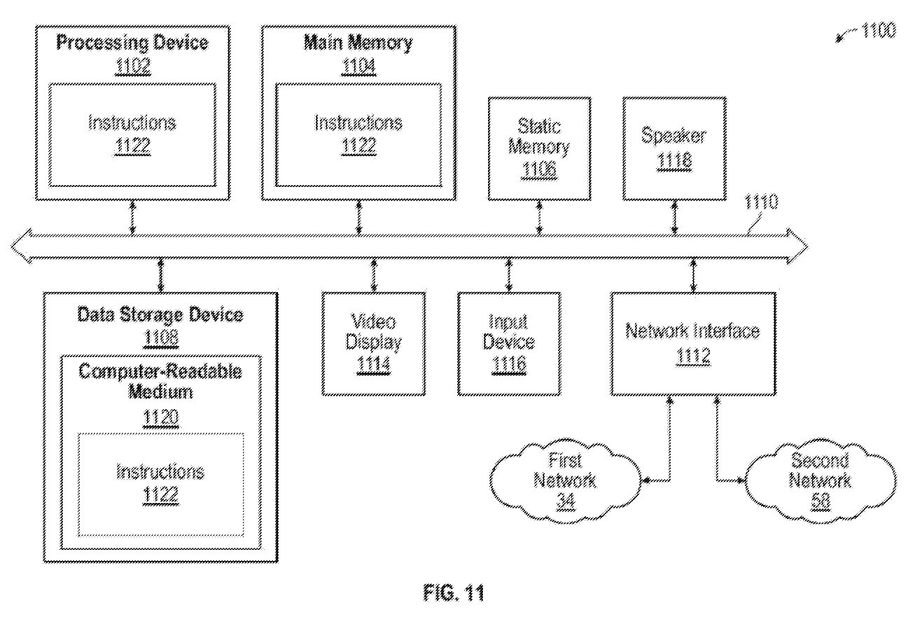

FIG. 11 shows an example computer system 1100 which can perform any one or more of the methods described herein, in accordance with one or more aspects of the present disclosure. In one example, computer system 1100 may include a computing device and correspond to the assistance interface 94, reporting interface 92, supervisory interface 90, clinician interface 20, server 30 (including the AI engine 11), patient interface 50, ambulatory sensor 82, goniometer 84, treatment apparatus 70, pressure sensor 86, or any suitable component of FIG. 1. The computer system 1100 may be capable of executing instructions implementing the one or more machine learning models 13 of the artificial intelligence engine 11 of FIG. 1. The computer system may be connected (e.g., networked) to other computer systems in a LAN, an intranet, an extranet, or the Internet, including via the cloud or a peer-to-peer network. The computer system may operate in the capacity of a server in a client-server network environment. The computer system may be a personal computer (PC), a tablet computer, a wearable (e.g., wristband), a set-top box (STB), a personal Digital Assistant (PDA), a mobile phone, a camera, a video camera, an Internet of Things (IoT) device, or any device capable of executing a set of instructions (sequential or otherwise) that specify actions to be taken by that device. Further, while only a single computer system is illustrated, the term “computer” shall also be taken to include any collection of computers that individually or jointly execute a set (or multiple sets) of instructions to perform any one or more of the methods discussed herein.

The computer system 1100 includes a processing device 1102, a main memory 1104 (e.g., read-only memory (ROM), flash memory, solid state drives (SSDs), dynamic random access memory (DRAM) such as synchronous DRAM (SDRAM)), a static memory 1106 (e.g., flash memory, solid state drives (SSDs), static random access memory (SRAM)), and a data storage device 1108, which communicate with each other via a bus 1110.

Processing device 1102 represents one or more general-purpose processing devices such as a microprocessor, central processing unit, or the like. More particularly, the processing device 1102 may be a complex instruction set computing (CISC) microprocessor, reduced instruction set computing (RISC) microprocessor, very long instruction word (VLIW) microprocessor, or a processor implementing other instruction sets or processors implementing a combination of instruction sets. The processing device 1102 may also be one or more special-purpose processing devices such as an application specific integrated circuit (ASIC), a system on a chip, a field programmable gate array (FPGA), a digital signal processor (DSP), network processor, or the like. The processing device 1102 is configured to execute instructions for performing any of the operations and steps discussed herein.

The computer system 1100 may further include a network interface device 1112. The computer system 1100 also may include a video display 1114 (e.g., a liquid crystal display (LCD), a light-emitting diode (LED), an organic light-emitting diode (OLED), a quantum LED, a cathode ray tube (CRT), a shadow mask CRT, an aperture grille CRT, a monochrome CRT), one or more input devices 1116 (e.g., a keyboard and/or a mouse or a gaming-like control), and one or more speakers 1118 (e.g., a speaker). In one illustrative example, the video display 1114 and the input device(s) 1116 may be combined into a single component or device (e.g., an LCD touch screen).

The data storage device 1116 may include a computer-readable medium 1120 on which the instructions 1122 embodying any one or more of the methods, operations, or functions described herein is stored. The instructions 1122 may also reside, completely or at least partially, within the main memory 1104 and/or within the processing device 1102 during execution thereof by the computer system 1100. As such, the main memory 1104 and the processing device 1102 also constitute computer-readable media. The instructions 1122 may further be transmitted or received over a network via the network interface device 1112.

While the computer-readable storage medium 1120 is shown in the illustrative examples to be a single medium, the term “computer-readable storage medium” should be taken to include a single medium or multiple media (e.g., a centralized or distributed database, and/or associated caches and servers) that store the one or more sets of instructions. The term “computer-readable storage medium” shall also be taken to include any medium that is capable of storing, encoding or carrying a set of instructions for execution by the machine and that cause the machine to perform any one or more of the methodologies of the present disclosure. The term “computer-readable storage medium” shall accordingly be taken to include, but not be limited to, solid-state memories, optical media, and magnetic media.

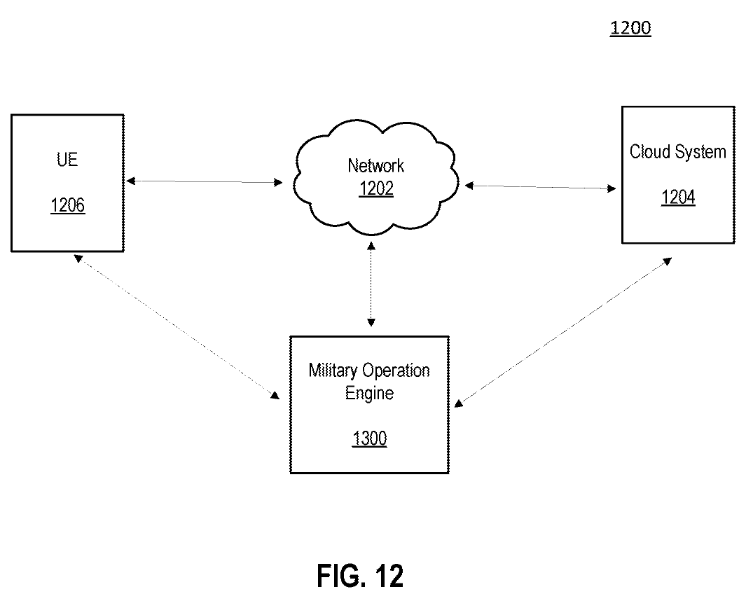

Turning now to FIG. 12, system (or framework) 1200 is depicted which includes UE 1206 (e.g., a client device), network 1202, cloud system 1204 and military operation engine 1300.

As discussed herein, the disclosed system 1200 provides for the automatic and computerized assignment of military operations, which effectively avails assigned users access to the securely held information siloed until such assignment. According to some embodiments, system 1200 can comparatively analyze an ops sheet of a military operation and profile data related to a user(s), and automatically determine whether a particular user(s) is capable of performing the operation (and/or the sub-tasks that are included). System 1200, as discussed below, provides a computerized platform that selects users for highly specific tasks based on the users’ analyzed skill sets, and performs computerized determinations regarding how such users are predicted to perform using those skill sets. System 1200, therefore, provides a new platform for military operations to be based, strategized, assigned and executed.

As illustrated in FIG. 12, UE 1206 can be any type of device, such as, but not limited to, a mobile phone, tablet, laptop, personal computer, sensor, Internet of Things (IoT) device, autonomous machine, the treatment apparatus of FIG. 2 (discussed supra) and any other device equipped with a cellular or wireless or wired transceiver.

In some embodiments, UE 1206 can represent a combination of devices, where UE 1206 is communicatively coupled to another peripheral device (not shown). For example, in some embodiments, UE 1206 can be a smartphone of a user, and the smartphone is paired with and connected to a wearable device (e.g., a smart watch).

Network 1202 can be any type of network, such as, but not limited to, a wireless network, cellular network, the Internet, and the like (as discussed above). As discussed herein, network 1202 can facilitate connectivity of the components of system 1200, as illustrated in FIG. 12.

Cloud system 1204 can be any type of cloud operating platform and/or network-based system upon which applications, operations, and/or other forms of network resources can be located. For example, system 1204 can correspond to a service provider, network provider, content provider and/or medical provider, or some combination thereof, from where services and/or applications can be accessed, sourced or executed. In some embodiments, cloud system 1204 can include a server(s) and/or a database of information which is accessible over network 1202. In some embodiments, a database (not shown) of system 1204 can store a dataset of data and metadata associated with local and/or network information related to a user(s) of UE 1206, users and the UE 1206, and the services and applications provided by cloud system 1204 and/or military operation engine 1300.

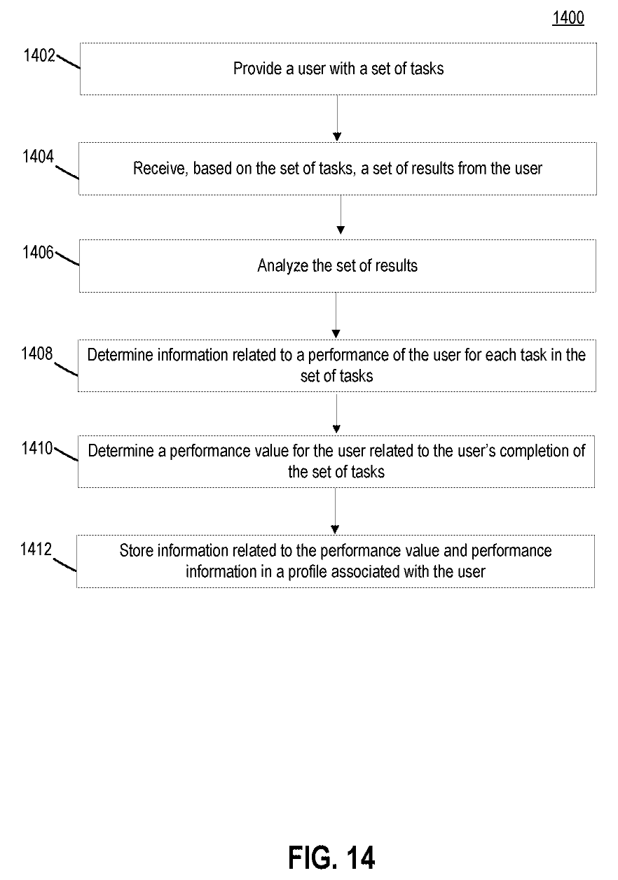

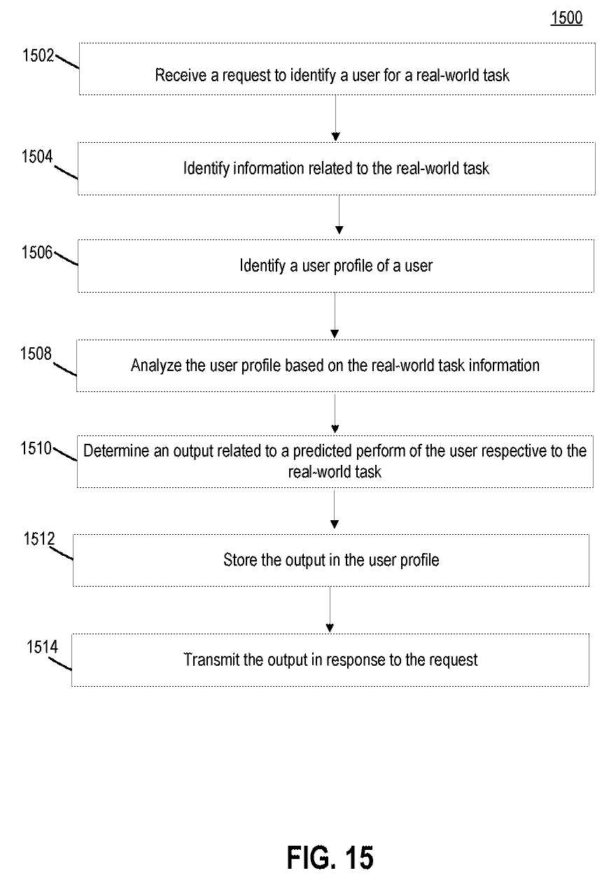

Military operation engine 1300, as discussed below in more detail, includes components for dynamically and automatically determining whether a user is physically, mentally and/or emotionally fit (e.g., possesses the necessary level of fitness across various domains of fitness) for a military operation, inclusive of each sub-task that is required for the operation to be deemed completed. In some embodiments, engine 1300 can be operated to identify users who are to be identified for a mission (or part of a mission), and in some embodiments, engine 1300 can be operated to determine whether a selected user is fit for a mission (or part of a mission), as discussed herein. Embodiments of how these operations of engine 1300 are performed, among others, are discussed in more detail below in relation to FIGS. 14 and 15.

According to some embodiments, military operation engine 1300 can be a special purpose machine or processor and could be hosted by a device on network 1202, within cloud system 1204 and/or on UE 1206. In some embodiments, engine 1300 can be hosted by a peripheral device connected to UE 1206 via a wired and/or wireless mechanism.

According to some embodiments, military operation engine 1300 can function as an application provided by cloud system 1204. In some embodiments, engine 1300 can function as an application installed on UE 1206. In some embodiments, such application can be a web-based application accessed by UE 1206 over network 1202 from cloud system 1204 (e.g., as indicated by the connection between network 1202 and engine 1300, and/or the dashed line between UE 1206 and engine 1300 in FIG. 12). In some embodiments, engine 1300 can be configured and/or installed as an augmenting script, program or application (e.g., a plug-in or extension) to another application or program provided by cloud system 1204 and/or executing on UE 1206.

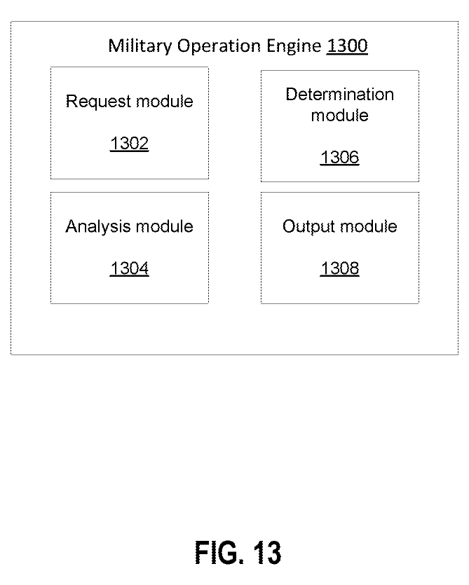

As illustrated in FIG. 13, according to some embodiments, military operation engine 1300 may include request module 1302, analysis module 1304, determination module 1306 and output module 1308. It should be understood that the engine(s) and modules discussed herein are non-exhaustive, as additional or fewer engines and/or modules (or sub-modules) may be included in the embodiments of the systems and methods discussed. More detail of the operations, configurations and functionalities of engine 1300 and each of its modules, and their roles within embodiments of the present disclosure, will be discussed below with reference to at least FIGS. 14-17.

Turning now to FIGS. 14-15, disclosed are embodiments for a computerized framework that dynamically determines (or identifies) capable users for military operations, and securely provides electronic information and/or access to devices and/or accounts of such determined users.

According to some embodiments, as discussed herein, FIG. 14 details embodiments for compiling profiles (or EMRs, discussed supra) for users, where such profiles may include information related to, but not limited to, the traits, attributes, characteristics, identities, capabilities, intentions, and the like, for each user. For example, to ascertain the capabilities of particular soldiers within particular units, engine 1300 can execute program logic in connection with the steps of FIG. 14 . Based on this, as detailed in FIG. 15, to determine which user(s) is fit for a particular mission, engine 1300 operates to leverage the profile data of each user.

While the discussion herein related to Processes 1400 and 1500 of FIGS. 14 and 15, respectively are discussed in relation to an individual user, the applicability of these processes can be extended to any number of users (e.g., a small test group, a particular demographic or psychographic, a military unit, a military division, and the like, for example) without departing from the scope of the instant disclosure.

Turning to FIG. 14, Process 1400 is disclosed which details non-limiting sample embodiments of building profiles for a set of users. As discussed herein, each profile is compiled based on analysis of how a user responds to a set of test tasks (and/or previously supplied tasks), whereby the profile can indicate performance metrics related to the users’ past, present and/or expected capabilities (e.g., how physically fit the user is, how strong the user’s mental facilities are, and how emotionally mature the user is, for example), as discussed below.

According to some embodiments, Steps 1402-1404 of Process 1400 can be performed by request module 1302 of military operation engine 1300; Step 1406 can be performed by analysis module 1304; Steps 1408-1410 can be performed by determination module 1306; and Step 1412 can be performed by output module 1412.

Process 1400 begins with Step 1402 where a set of tasks are provided to a user. As discussed above, the user can be understood to be a soldier and/or other highly trained individual with a specialized skill set for performing highly specialized tasks. In some embodiments, the set of tasks can include, but are not limited to, physical activities, intelligence tests, emotional tests, and the like, or some combination thereof.

For example, Step 1402 can involve requesting a user to run a mile in under 5 minutes while carrying all his gear and, then at the completion of the mile, to answer 25 specifically selected questions related to a type of military operation.

According to some embodiments, the set of tasks can include a physical activity, a digital activity, a physical activity and digital activity, and the like, or some combination thereof. In some embodiments, the tasks can correspond to a treatment plan, as discussed supra.

In Step 1404, a set of results are provided by the user and received by engine 1300. According to some embodiments, the set of results can be embodied or formatted as an individual data structure of a file created based on the monitored tracking of the user’s progress for each of the tasks of Step 1402. In some embodiments, at least a portion of the data constituting the set of results can be generated, captured or otherwise identified by UE 1206. Thus, Step 1404 can involve a file being generated, and continually or continuously updated as a user progresses through the tasks (or at the completion of a portion of or all of the tasks), where the file can indicate performance values for the user respectiveto the task.

In Step 1406, engine 1300 can analyze the set of results. In some embodiments, the analysis can involve engine 1300 parsing the set of results data structure/file, and determining (or mining for) information that indicates how the user performed on each of the individual tasks of the set of tasks.

According to some embodiments, the analysis performed in Step 1406 can involve the set of results being used as inputs to any type of known or to be known ML/AI computational analysis algorithm, technology, mechanism or classifier, such as, but not limited to, a neural network (e.g., artificial neural network analysis (ANN), convolutional neural network (CNN) analysis, and the like), computer vision, cluster analysis, data mining, Bayesian network analysis, Hidden Markov models, logical model and/or tree analysis, and the like.

It should be understood that while the disclosure herein will be discussed with reference to engine 1300 executing a ML algorithm, technique, technology or mechanism, it should not be construed as limiting, as any type of trainable software, technology and/or executable computer/program-logic can be utilized in a similar manner without departing from the scope of the instant disclosure. For example, an algorithm can be any type of known or to-be-known ML algorithm, AI algorithm, greedy algorithm, recursive algorithm, and the like, or some combination thereof. Moreover, the ML algorithm can be any type of known or to-be-known support vector machine or logistic regression predictive modelling, and the like.

Moreover, engine 1300 can be trained based on training data sets that include information related to military missions, user profile information, and the like, as well as recursive results from the processing of Processes 1400 and 1500, discussed herein, thereby enabling accurate and up-to-date ML processing for purposes of performing the analysis and determinations discussed herein.

As a result of the analysis performed by engine 1300 in Step 1406, engine 1300 can perform Step 1408 where information related to a performance of the user for each task in the set of tasks is determined. That is, engine 1300 outputs, via the ML analysis of the set of results, a determination of how the user performed on each task. According to some embodiments, the determination can be a binary value that indicates whether a task in the set of tasks (from Step 1402) was completed or not (e.g., 1 or 0). In some embodiments, the determination can also provide a degree, value or metric (or other quantifiable indicator) that indicates how the user performed. This can involve setting completion to a baseline threshold value, and to what degree the user surpassed or failed to satisfy the threshold. For example, as discussed above, if the user were to run a 5-minute mile, the threshold can be viewed as 5 minutes, and any time less than 5 minutes would then correspond to completing the task, while any time longer than 5 minutes would correspond to failing the task. Moreover, the threshold can have ranges that correspond to outlier performance. For example, if the user’s time were more than 30 seconds faster than 5 minutes, then this can be indicated in the determination. Such outlier status can be set by an administrator, based on how other users performed, based on a type of task, and/or any other type of criteria that can construe the difficulty of a task.

In Step 1410, engine 1400 can determine a performance value for the user wherein the performance value is related to the completion of the set of tasks. That is, in some embodiments, Step 1410 provides an indicator that relays how the user performed overall for the set of tasks. In some embodiments, Step 1410 can involve an indicator for each task, as provided for above in relation to Step 1408. In some embodiments, the determination of Step 1410 can be a by-product of engine 1300 executing the ML/AI mechanisms on the determined information from Step 1408 such that a tangible value is output for purposes of measuring indicators of a user’s performance. For example, Step 1410 can involve creating an n-dimensional vector where nodes on the vector correspond to values of a user’s performance for specific tasks.

According to some embodiments, Step 1410 can involve the creation of a data structure or file for the user that provides an indication of the determined value for the user. In some embodiments, the creation of the structure/file can be encrypted or otherwise secured via privacy enhancing technologies (PETs), so that it is incapable of being modified or changed by a malicious actor (e.g., for example, by having security enabled to encrypt the data or store the data on a blockchain, for example).

In Step 1412, engine 1300 can store this information in a profile (or EMR, supra) of the user. The stored information can include, but may not be limited to, the determined information (from Step 1408) and/or the determined value (from Step 1410), as well as other forms of cohort data (supra), and/or any other type of identifying user data, and the like.

By way of a non-limiting example, a user profile can include characteristics of a user that can include, but are not limited to, a personal or other identifier, demographic information, geographic information, behavioral history, user interests, user preferences or settings, history of task completion, rank, military unit, as association with the United States’ Department of Defense (DOD), an association with another country’s governmental organization responsible for defense of the country, biometric information, pain tolerance information, treatment plan information, training metrics, psychological information, intelligence quotient (IQ) scores, emotional quotient (EQ) scores, classification testing scores and user-provided feedback, and the like, or some combination thereof.

In some embodiments, Step 1412 can involve creating a new profile for a user; and in some embodiments, Step 1412 can involve updating an existing profile for the user.

Turning to FIG. 15, Process 1500 provides non-limiting sample embodiments for selecting a user for a military mission. As discussed above, a military mission is a highly specialized task that requires a specifically trained individual (or set of individuals) to perform a set of tasks in order for the mission to be completed. For example, a group of Navy Seals can be identified as comprising the types of soldiers required for a mission, and Process 1500 can operate to enable determining which Seal(s) is fit for the mission.

According to some embodiments, Steps 1502-1506 of Process 1500 can be performed by request module 1302 of military operation engine 1300; Step 1508 can be performed by analysis module 1304; Step 1510 can be performed by determination module 1306; and Steps 1512-1514 can be performed by output module 1308.

Process 1500 begins with Step 1502 where engine 1300 receives a request to identify a user for a real-world task. In some embodiments, the request can be automatically generated based on the detection of real-world events that trigger a type of mission to be required. In some embodiments, the request can be provided by a requesting user (e.g., a requestor such as, for example, an officer, general, and the like). In some embodiments, the request can be subject to security clearance of a user. Such a technique ensures that only users (e.g., a General) with proper security clearances can make a request.

In some embodiments, the real-world task can correspond to a military operation, which one of skill in the art would understand requires a specialized set of capabilities operable at a level surpassing a threshold associated with civilian operations. In some embodiments, a real-world task can include a set of activities required to be completed in order for the real-world task to be considered completed. For example, such activities may include, without limitation, driving a military helicopter, operating a military-type drone, commanding a submarine, navigating a rural and/or urban assault vehicle and/or terrain, and the like, and/or any other type of activity that requires specialized training that military-type personnel have undergone.

In some embodiments, the request of Step 1502 can identify a user so that a determination can be made regarding whether that user is fit for the real-world task (e.g., that particular mission). In some embodiments, the request of Step 1502 can include a sub-request to identify a user from a set of users. Each user in the set may be comparatively analyzed to identify and rank those users who are most suited (or the best fit), as discussed herein.

In Step 1504, engine 1300 can identify information related to the real-world task. In some embodiments, the engine 1300 may parse the request and extract information relating to the real-world task. In some embodiments, the real-world task can be embodied as a secure file that requires an access key/token to unlock. In such embodiments, in order to perform the steps of Process 1500, engine 1300 can retrieve the key/token, and gain access to the file.

In some embodiments, the real-world task can be formatted as a digital file, where information related to each task (or sub-task – e.g., the ops sheet) can be provided via, but not limited to, text, audio, video, multimedia, AR/VR, global positioning system (GPS) data, coordinates, smart contracts, and the like, and/or via any other type of known or to-be-known data format.

In Step 1506, engine 1300 can identify a user profile of a user. According to some embodiments, the profile of the user identified is the profile that was stored (e.g., created or updated) as per the processing of Process 1400, supra. In some embodiments, the profile can also include third-party profiles (e.g., social networking profiles, for example), which can be used to supplement the capability characteristics of the user. Accordingly, one or more APIs associated with a third-party service (e.g., social networking platform) may be accessed to obtain the third-party profiles.

In some embodiments, Step 1506 can involve retrieving or extracting the profile and/or profile information (e.g., characteristics) for the user from a database of user profiles (e.g., EMRs). As such, in Step 1506, engine 1300 is capable of retrieving information of the user that relates to or specifies a skillset of the user. The skillset of the user may correspond to at least one of a set of physical capabilities, a set of intellectual capabilities, and/or a set of emotional capabilities. In some embodiments, these capabilities can be partitioned to indicate current capabilities, past capabilities (according to a predetermined time period), or some combination thereof.

In Step 1508, engine 1308 performs a comparative analysis of the user profile information (from Step 1506) and the real-world task information (from Step 1504) via a predictive ML model that enables engine 1300 to determine (or predict) an expected performance of the user in each activity of the real-world task.

According to some embodiments, the analysis of Step 1508 can be performed via similar AI/ML mechanisms as discussed above in relation to Steps 1406-1408 of Process 1400 of FIG. 14. Therefore, as a result of the analysis of Step 1508, engine 1300 can determine an output that indicates the predicted performance of the user in the real-world task. According to some embodiments, the determined (or generated) output can include information related to a status of the real-world task and a performance level associated with the status. In some embodiments, the status of the real-world task can correspond to an indication of whether all or at least a portion of the set of activities is determined as capable of being performed by the user. In some embodiments, the performance level can correspond to a metric associated with the expected performance of the user. For example, in a similar manner as discussed above in relation to degree of success/failure via an outlier performance, the performance level can indicate a value as to how, and to which degree, the user is expected to surpass or fail the task (e.g., run a mile 2 minutes longer than a 5-minute threshold, for example).

In Step 1512, engine 1300 causes the output to be stored in the profile of the user. Thus, the profile of the user (identified in Step 1506) can be updated based on the processing of Process 1500, thereby providing a more accurate and up-to-date assessment of the user’s capabilities. The user’s capabilities can be leveraged for subsequent performance projections/predictions.

In Step 1514, engine 1300 can transmit (or communicate) the output. In some embodiments, the output can be sent to a device or account of a user that triggered the request (e.g., the requestor). In some embodiments, the output can be posted to a portal for access by another user(s) who has a certain level of security clearance or credentials. In some embodiments, a notification of the output can be transmitted to user(s). The notification may indicate that results are ready to be accessed via a secure account. In some embodiments, the output can also be sent to the user so that the user can gauge their strengths and/or weaknesses as they relate to the activities of the real-world task.

In some embodiments, the transmission of Step 1514 can involve the secure communication protocols discussed below at least in relation to Processes 1600 and 1700, and Steps 1620 and 1714, respectively.

In some embodiments, the output can be fed back to engine 1300 so as to recursively train the ML models executing therein.

In some embodiments, the output can be sent to ML model 13 (of FIG. 6) so that a treatment plan 602 can be created to improve the user’s expected performance, as discussed above. For example, ML model 13 (and/or engine 1300) can analyze the current capabilities of the user as compared to baseline threshold values of each activity of the real-world task. Based on the comparison, the ML model 13 may generate a treatment plan so the user can improve specific types of capabilities (or skillsets).

In some embodiments, the output can include displayable results that graphically indicate measures related to how the user performs compared to, but not limited to, thresholds for each activity, measures related to how other users perform, measures related to how the user performed in the past (according to certain time periods), and the like, or some combination thereof.

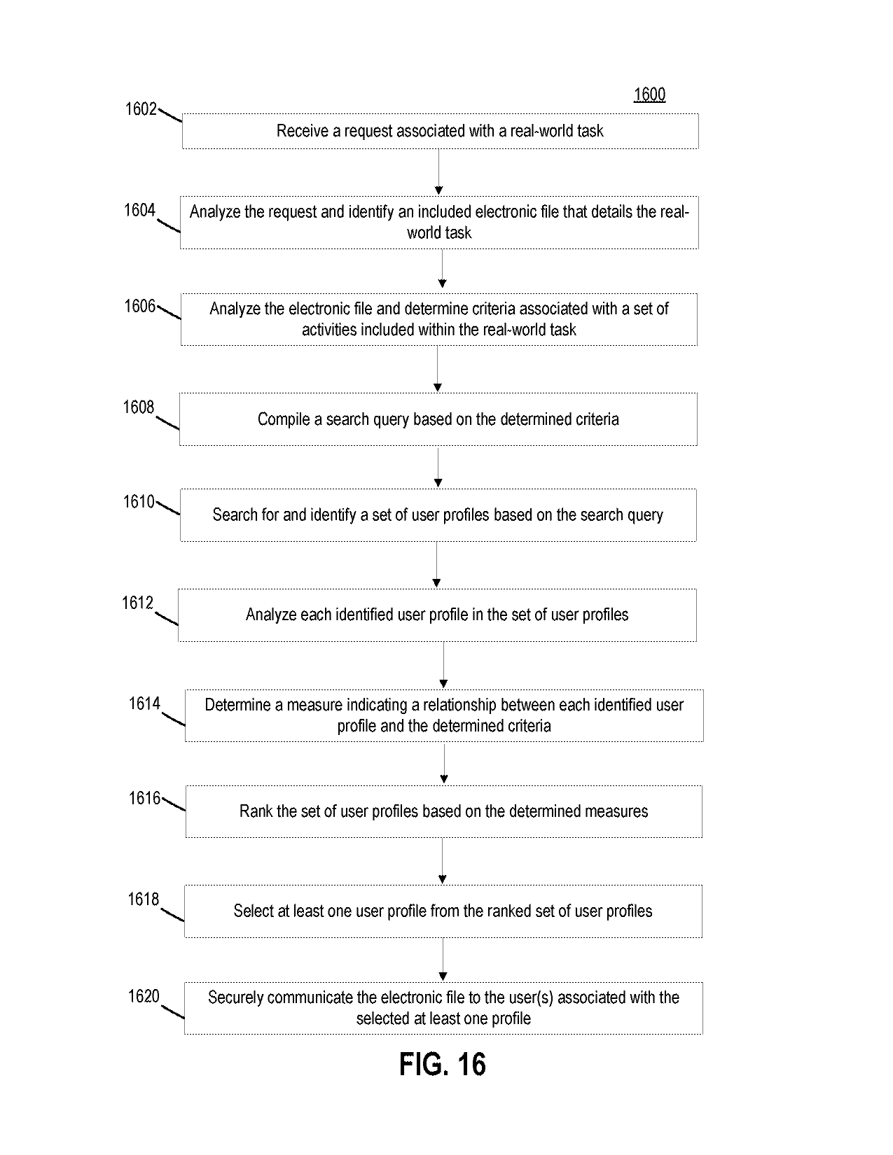

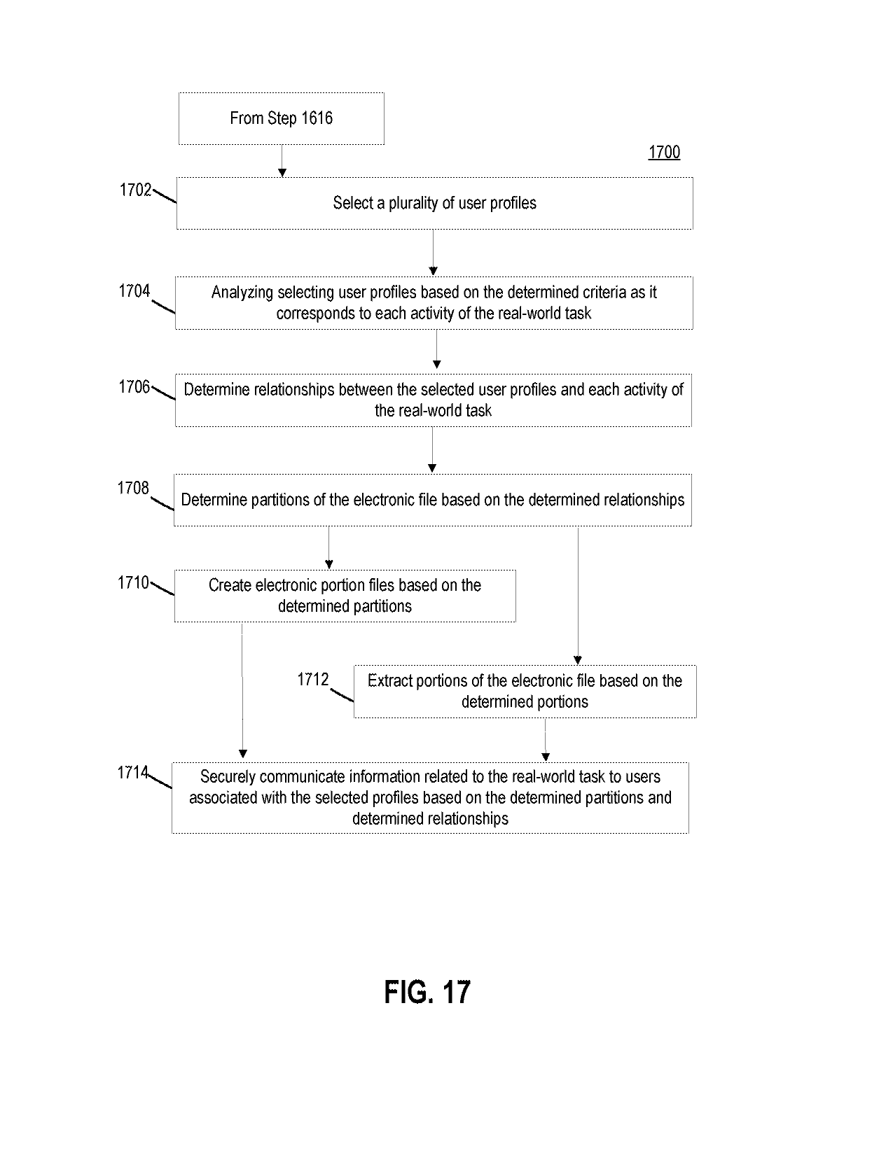

Turning to FIGS. 16 and 17, Processes 1600 and 1700, respectively, provide non-limiting embodiments for a computerized framework that leverages AI/ML mechanisms to assign military operations to selected individuals. The disclosed framework (embodied via system 1200, supra) is configured to comparatively analyze an ops sheet of a military operation and profile data related to a user(s), and automatically determine a user(s) who is optimal for the operation. That is, the framework can determine or otherwise identify (or locate) which user or users possess the physical, intellectual, emotional and/or psychological capabilities to accurately and efficiently, with respect to real-world and electronic resources, perform and complete the operation (e.g., complete the mission in the most resource- and financially-economical manner possible, or in any other measurable manner possible).

The disclosed framework, therefore, provides a computerized platform that selects users for highly specific tasks based on the users’ analyzed skill sets and based on computerized determinations of how such users are predicted to perform using those skill sets. Upon selectively assigning an operation to a user(s) determined “fit” for the operation, the framework can securely and/or confidentially provide access to information related to the operation.

In FIG. 16, Process 1600 provides non-limiting sample embodiments for receiving a request to identify individuals for a mission and for identifying a best-suited individual(s), where the identifications are based on the requirements of the mission in conjunction with the skills and capabilities of a pool of available individuals.

For example, if a mission is to fly a jet to location X and drop cargo, the pool of users can be identified as a set of lieutenants in the Air Force™. From there, each lieutenant’s capabilities and skills are analyzed to determine which lieutenant is best suited for the mission. Upon the identification of a candidate for the mission, the framework enables secure access to the confidential data related to the mission.

While the discussion herein related to Process 1600 of FIG. 16 is discussed in relation to an individual user, it should be understood that the applicability of this process can be extended to any number of users (e.g., a small test group, a particular demographic, a military unit, a military division, and the like, for example) without departing from the scope of the instant disclosure.

According to some embodiments, Step 1602 of Process 1600 can be performed by request module 1302 of military operation engine 1300; Steps 1604-1606 and 1612 can be performed by analysis module 1304; Steps 1608-1610 and 1614-1616 can be performed by determination module 1306; and Steps 1618-1620 can be performed by output module 1308.

Process 1600 begins with Step 1602, where a request is received in relation to a real-world task. As discussed above, the real-world task can correspond to a military mission, and the request can originate from a requestor and/or be automatically triggered from an application or based on a detected news story or current event, and the like.

According to some embodiments, the request can include, but is not limited to, information indicating a number of users required for a mission, details of the mission, an identity of a requested user(s), an identity of a requesting user, equipment needed for the mission, a time period for the mission, security clearances needed for the mission, and the like, or some combination thereof.

In Step 1602, engine 1300 can analyze the request and identify an electronic file(s) that details the real-world task (e.g., provides mission details or a set of activities to be performed as part of the mission). In some embodiments, the request can be a message that includes the electronic file. In some embodiments, the request can comprise a pointer to a location where the electronic file can be securely retrieved. As such, according to some embodiments, Step 1602 can involve engine 1300 parsing the request and identifying the electronic file.

In some embodiments, the electronic file can be a type of format, configuration, size and/or subject to any type of security (e.g., any type of a privacy enhancing technology (PET) or security enhancing technology (SET)), and the like, or some combination thereof. As discussed below, in order for a selected user(s) to access the file, unlocking/security steps may be required by their device and/or engine 1300.

In Step 1606, engine 1300 can analyze the electronic file and determine criteria associated with the set of activities of the real-world task. According to some embodiments, a particular criterion may correspond to a required characteristic a user must have to perform a specific activity of the real-world task. For example, if the task, and as a result an activity included therein, requires flying an airplane, then a user must possess a piloting skillset for a specific type of military aerial vehicle.

According to some embodiments, as discussed above, engine 1300 can perform the analysis and determination according to any type of known or to-be-known ML/AI computational analysis algorithm, technology, mechanism or classifier, such as, but not limited to, a neural network (e.g., artificial neural network analysis (ANN), convolutional neural network (CNN) analysis, and the like), computer vision, cluster analysis, data mining, Bayesian network analysis, Hidden Markov models, logical model and/or tree analysis, and the like.

In Step 1608, engine 1300 can then compile a search query based on the determined criteria. For example, the query can include indicators of the types of capabilities required for each activity of the real-world task. In some embodiments, the query can be formatted via any type of known or to-be-known format including, but not limited to, a text query, Boolean string, n-dimensional vector, and the like, or some combination thereof. In some embodiments, the query can be formatted according to a format or structure of the database upon which engine 1300 will be searching.

In some embodiments, the search can be performed with respect to a database of military user profile information, as discussed above; and/or can be subject to third-party databases (e.g., social networking systems) so as to mine user profile information from remote locations, as discussed above.

In Step 1610, engine 1300 executes a search via the compiled search query. As a result of the search, engine 1300 can identify a set of user profiles that comply with the determined criteria. In some embodiments, engine 1300 can analyze each user profile in the user database, and determine which profiles include characteristics that indicate a skillset or capability that maps to the determined criteria. In some embodiments, such determination can be a result of a similarity sort or comparison algorithm or mechanism executed by engine 1300, which enables the identification of similar data items within a profile and a query. In some embodiments, such similarity can be subject to a similarity threshold, whereby upon a data item in a profile matching at least to a threshold satisfying value the criteria in the query, then that user profile can be identified as having a criterion at least satisfying at least a portion of the search.

According to some embodiments, the search and determination executed in Step 1610 can involve engine 1300 utilizing any type of known or to-be-known sorting or comparison algorithm, technique, mechanism or technology, such as, but not limited to, a comparison sort, string search, quicksort, cosine similarity, word2vec or doc2vec, Euclidean distance, and the like, or some combination thereof.

Thus, as a result of Step 1610, a set (or list) of user profiles is identified. These user profiles include information that at least indicates a capability of an associated user that maps to a determined criterion of the real-world task (e.g., the user has the skills to perform the task/activity).

In Step 1612, engine 1300 can then analyze the user profiles, and in Step 1614, determine a measure (also referred to as a “mission score”) that indicates a relationship between each identified user profile and the determined criterion or criteria. That is, engine 1300 can determine how “on point” or inclusive the user profile is in containing characteristics that indicate a skillset or capability for performing an activity or activities of the real-world task. In some embodiments, engine 1300 can determine such measures via execution of the AI/ML and/or comparative analysis algorithms discussed above.

According to some embodiments, each user profile in the identified search result (and ranked, as provided for below) has a measure / mission score that satisfies a mission threshold corresponding to a minimum set of characteristics or minimum matching characteristic to a criterion.

According to some embodiments, Step 1610’s search and determination can result in the determined measure discussed in relation to Step 1614. Thus, as a result of engine 1300 executing Step 1610, the measure determined from Step 1614 for each user profile can already be computed (i.e., Steps 1612-1614 can be sub-steps of Step 1610).

In Step 1616, engine 1300 can therefore rank the identified user profiles (from Step 1610). According to some embodiments, such ranking can be based on the determined measure from Step 1614. For example, those profiles with scores greater than others can be ranked higher in the ranked set of user profiles.

In some embodiments, such ranking can further involve a weighting feature, such that as a user profile has more characteristics per activity and/or task (e.g., matching more activities within the task), then that profile can be subject to a weight proportional to the number of activities to which it corresponds. For example, if user profile A indicates that user A is a Navy Seal who can operate machine X, and user profile B indicates that user B is a Marine who can operate machine X, then user A’s matching may be weighted more if the mission requests Navy Seal training in addition to the capability to operate machine X.

In Step 1618, engine 1300 can effectuate a selection of at least one user profile from the ranked set. In some embodiments, the number of selected profiles can correspond to how many users are identified as being needed in the request. In some embodiments, the selection of a user profile can be performed automatically, without user input, by identifying the highest ranked user profile. In some embodiments, such automatic selection can be based on ML classifier analysis executed by engine 1300, which can be performed in manner similar to that discussed above. In some embodiments, feedback can be received that alters how the engine 1300 can select user profiles; for example, if two user profiles are ranked/scored within a predetermined range of each other, then engine 1300 can cause a notification to be sent to a specific user (e.g., an officer or general, for example) to request that a selection be made between two otherwise comparable candidates. And, in some embodiments, a user can provide input to select from a display of the ranked set a user profile(s).

In Step 1620, engine 1300 can securely communicate the electronic file to a device and/or account of the user(s) associated with selected profile(s). In some embodiments, such communication (or transmission) enables the user to access the electronic file. In some embodiments, such access can include classified and protected information related to the real-world task. In some embodiments, such access can include granting permissions that enable the user to access (and/or use) specific equipment for performing the real-world task. In some embodiments, as discussed above, such secure communication can involve PET or SET.

According to some embodiments, the electronic file can be accessed (or opened) by a user via unlocking steps to unlock the features of the file and/or the message or location that houses the file. In some embodiments, the file may be encrypted (and/or message/location may be subject to encryption or added security (e.g., 2-step verification, for example); therefore, decryption via the security key may be required to access the file/message. In some embodiments, the key can be provided to the selected user(s), and in some embodiments, the file may be decrypted and then securely transmitted to the selected user(s).

In some embodiments, information related to the secure communication, the selected user, their profile information and the real-world task can be fed back to engine 1300 so as to recursively train the ML models executing therein.

In FIG. 17, Process 1700 provides non-limiting sample embodiments where a mission is assigned to a group of users (e.g., a team, squad or unit, for example), and each member of the team is assigned a specific portion of the mission. In some embodiments, as discussed below, members of the group may only be provided access to portions of the mission for which they are assigned. This can effectively realize increased security as users are provided information on a “need to know” basis even on missions where they are working in concert with other users. In some embodiments, some users may receive information only upon the determined completion of a preceding part.

According to some embodiments, Steps 1702 of Process 1700 can be performed by request module 1302 of military operation engine 1300; Step 1704 can be performed by analysis module 1304; Steps 1706-1708 can be performed by determination module 1306; and Steps 1710-1714 can be performed by output module 1308.

Process 1700 begins from Step 1616 of Process 1600 where a ranked set of user profiles is provided. Additionally, in the embodiments discussed herein, the request for performance of a real-world task (from Step 1602 of Process 1600, supra) has indicated that a predetermined number of users (e.g., more than 1) is required for the mission.

In Step 1702, engine 1300 selects a plurality of user profiles. This can be performed in a similar manner as discussed above in relation to Step 1618 of Process 1600. As mentioned above, the request (from Step 1602) can indicate that a predetermined number of users is required for a real-world task and/or an activity included therein. Therefore, the selection of a plurality of user profiles can be based on that predetermined number.

By way of a non-limiting example, if the real-world task (or at least one activity included therein) involves sniper activity, this can involve at least two individuals: a gunman and spotter. Therefore, based on this, two user profiles can be identified.

As such, according to some embodiments, the identification and ranking of user profiles can correspond to a prospective team of users, whereby the selection performed in Step 1702 (and, in some embodiments, Step 1618) can correspond to the selection of an actual team to perform the real-world task. As such, the next processing of Process 1700 involves determining which selected users (i.e., members of the team) are to perform particular tasks (e.g., user A is the gunman, and user B is the spotter).

In Step 1704, engine 1300 can perform, based on the determined criteria as they correspond to each activity of the real-world task, an analysis on the selected user profiles. Such analysis can be performed via the ML/AI processing discussed above at least in relation to Steps 1606 and 1612, inter alia.

Based on the analysis of Step 1704, engine 1300 can then determine relationships between the selected user profiles and each activity of the real-world task, as in Step 1706. Such relationship determinations can be performed in a similar manner as discussed above in relation to at least Steps 1606 and 1614, inter alia. Indeed, engine 1300 can determine which user profiles have included therein information (e.g., characteristics indicating a type, quantity, rank and/or level of capability) that indicates that the associated user can perform an activity. For example, the selected user profile for the user with sniper training leads engine 1300 to determine a relationship with the gunman role, and the same for another user and the spotter role.

Thus, Step 1706 involves engine 1300 determining which members of the actual team are to be associated with performing each activity of the set of activities of the real-world task.

In Step 1708, based on the determined relationships from Step 1706, engine 1300 determines partitions of the electronic file. According to some embodiments, engine 1300 parses the electronic file based on the determined relationships and identifies each portion within the file that corresponds to particular activities.

According to some embodiments, Process 1700 can then proceed in three different ways: i) to Step 1710 for each determined partition; ii) to Step 1712 for each partition; or iii) to Step 1710 for a portion of the determined partitions, and, subsequently thereafter, to Step 1712 for a remaining portion of the determined partitions.

In some embodiments, engine 1300 can determine which is the next step(s) based on, but not limited to, bandwidth on a network, storage availability and/or processing power on the device executing engine 1300, preferences or settings of a network and/or administrator, security settings applied to the electronic file, the size of the file, the format of the file, and the like, or some combination thereof.

In Step 1710, engine 1300 can create electronic portion files based on the determined partitions. In some embodiments, this may involve extracting particular portions from the electronic file, then creating a new file to be sent to a user of a determined relationship.

For example, the portion of the electronic file to be sent to the user assigned as the gunman can be extracted and used as the content for a new electronic file that only includes that information (e.g., data/metadata for the particular gunman activity).

In Step 1712, engine 1300 can extract, based on the determined portions, portions of the electronic file, and rather than create a new file (as in Step 1710), the extracted content can be included as part of a message to be sent to the assigned user. In some embodiments, such extracted content can be posted to a secure portal and/or high security account or property associated with the assigned user/member – for example, an encrypted mail account of the user.

As a result of Step 1710, Step 1712 or Steps 1710 and 1712, engine 1300 then performs Step 1714 where the created file (from Step 1710) and/or secure message (from Step 1712) can be securely communicated to the users associated with the selected profiles. Such secure communication can be performed in a similar manner as discussed above at least in relation to Step 1620.

In some embodiments, information related to the secure communication, the selected users, their profile information and the real-world task can be fed back to engine 1300 so as to recursively train the ML models executing therein.

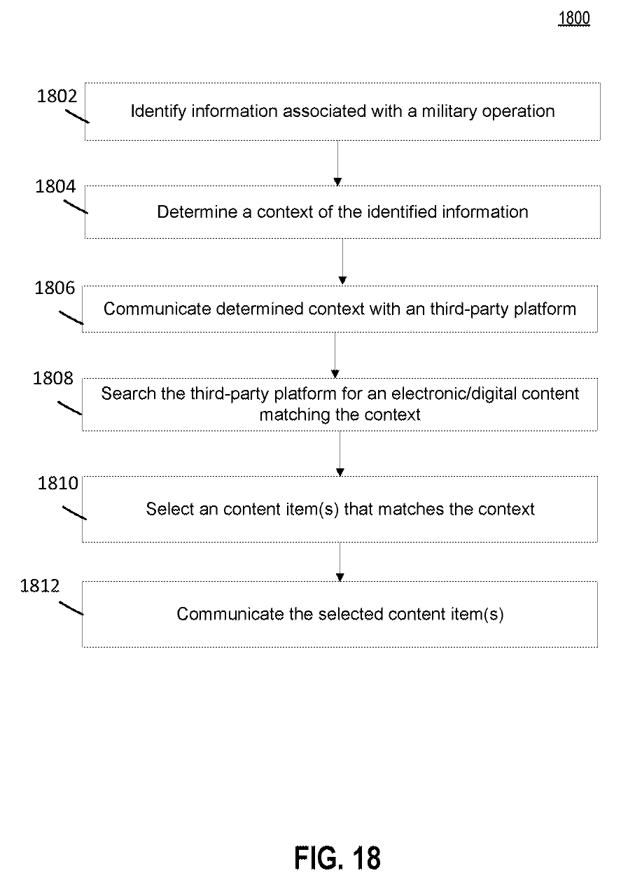

FIG. 18 is a workflow process 1800 for serving or providing related digital media content based on the information associated with a military mission, as discussed above at least in relation to FIGS. 14-17. In some embodiments, the provided content can be associated with or comprise advertisements (e.g., digital advertisement content). Such information can be referred to as “operation information” for reference purposes only.

As discussed above, reference to an “advertisement” should be understood to include, but not be limited to, digital media content that provides information provided by another user, service, third party, entity, and the like. Such digital ad content can include any type of known or to-be-known media renderable by a computing device, including, but not limited to, video, text, audio, images, and/or any other type of known or to-be-known multi-media. In some embodiments, the digital ad content can be formatted as hyperlinked multi-media content that provides deep-linking features and/or capabilities. Therefore, while the content is referred as an advertisement, it is still a digital media item renderable by a computing device, and such digital media item comprises digital content relaying promotional content provided by a network-associated third party.

In Step 1802, operation information is identified. This information can be derived, determined, based on or otherwise identified from the steps of Processes 800-1000 and 1400-1500, as discussed above in relation to FIGS. 8-10 and 1400-1700, respectively.

For purposes of this disclosure, Process 1800 will refer to single operation (and/or corresponding selected user(s)); however, it should not be construed as limiting, as any number of operations, sub-tasks and/or users can form such a basis, without departing from the scope of the present disclosure.

In Step 1804, a context is determined based on the identified operation information. This context forms a basis for serving content related to the operation information. For example, the context can be determined based on a location, required equipment and/or number of users for a particular mission. For example, if a mission requires a type of firearm X and type of footwear Y, then the context can correspond to X and Y.

In some embodiments, the identification of the context from Step 1804 can occur before, during and/or after the analysis detailed above with respect to at least Processes 800-1000 and 1400-1700, or it can be a separate process altogether, or some combination thereof.

In Step 1806, the determined context is communicated (or shared) with a content providing platform comprising a server and database (e.g., a content server and content database, and/or third-party server and associated third-party database, for example). Upon receipt of the context, the server performs (i.e., is caused to perform as per instructions received from the device executing the engine 1300) a search for a relevant digital content within the associated database. The search for the content is based at least on the identified context.

In Step 1808, the server searches the database for a digital content item(s) that matches the identified context. In Step 1810, a content item is selected (or retrieved) based on the results of Step 1808.

In some embodiments, the selected content item can be modified to conform to attributes or capabilities of a device, browser user interface (UI), page, interface, platform, application or method upon which a mission selection session will be initiated, continued and/or retained, and/or to the application and/or device for which selected user attributes are being displayed and/or rendered.

In some embodiments, the selected content item is shared or communicated via an application or browser a user is using to view mission selection results, as in Step 1812. For example, upon the identification of a particular user from Processes 1500, 1600 and/or 1700 for a mission, the selected content item can be provided which will enable the selection of the gear required for such mission.

In some embodiments, the selected content item is sent directly to a user computing device for display on a device and/or within the UI displayed on the device’s display (e.g., within the browser window and/or within an inbox of the high-security property). In some embodiments, the selected content item is displayed within a portion of the interface or within an overlaying or pop-up interface associated with a rendering interface displayed on the device.

Clause 1. A method comprising the steps of:

receiving, by a device, a request associated with a real-world task, the request comprising an electronic file, the electronic file comprising information related to the real-world task, the real-world task comprising a set of activities that are required to be completed;

parsing, by the device, the request;

identifying, by the device, based on the parsing of the request, the file;

analyzing, by the device, the file, and determining criteria associated with each of the set of activities, each determined criterion corresponding to a required characteristic a user must possess to complete a respective activity associated with the real-world task;

compiling, by the device, a search query based on the determined criteria;

searching, by the device, a database based on the search query, the database comprising a plurality of user profiles, each user profile comprising characteristics of a user;

determining, by the device, based on the search, a search result identifying a set of user profiles, each identified user profile comprising characteristics complying with the determined criteria;

ranking, by the device, the set of user profiles identified within the search result by ordering each identified user profile according to a measure of a respective user profile’s compliance with the determined criteria;

selecting from the ranked search result, by the device, a user profile; and

securely transmitting, via the device, data associated with the real-world task to an account of the user associated with the selected user profile.

Clause 2. The method of claim 1, wherein the transmission of the data enables the user to access at least one of classified and protected information related to the real-world task and to equipment for performing the real-world task.

Clause 3. The method of claim 1, wherein the searching of the database further comprises:

based on the determined criteria in the search query, analyzing, by the device, each user profile in the database;

determining, by the device, a mission score for each user profile, the mission score providing a measure of the user’s complying characteristics; and

based on the determined mission scores, generating, by the device, the ranked search result.

Clause 4. The method of claim 3, wherein each user profile in the ranked search result has a mission score satisfying a mission threshold corresponding to a minimum set of characteristics.

Clause 5. The method of claim 1, further comprising:

identifying, by the device, based on the search, another set of user profiles, each identified user profile in the other set corresponds to a user profile with characteristics satisfying at least one of the determined criteria associated with the set of activities encompassing the real-world task.

Clause 6. The method of claim 5, further comprising:

determining a prospective team of users based on the identified set of other user profiles; and

selecting, from the prospective team, an actual team to perform the real-world task, wherein the secure data is made available to each member of the actual team.

Clause 7. The method of claim 6, further comprising:

determining, by the device, which members of the actual team are to be associated with performing each activity of the set of activities of the real-world task;

partitioning according to each activity, by the device, the data; and

sending each member of the actual team a respective partitioned portion of the data.

Clause 8. The method of claim 1, wherein the characteristics of the user in the user profile are selected from a group of information related to the user consisting of: a personal or other identifier, demographic information, geographic information, behavioral history, history of task completion, rank, military unit, as association with the United States’ Department of Defense (DOD), an association with another country’s governmental organization responsible for defense of the country, biometric information, pain tolerance information, treatment plan information, training metrics, psychological information, intelligence quotient (IQ) scores, emotional quotient (EQ) scores, classification testing scores and user-provided feedback.

Clause 9. The method of claim 1, further comprising:

analyzing, by the device via a classifier model, the search result; and

automatically selecting, without user input, by the device, the user profile.

Clause 10. The method of claim 1, wherein the file included within the request is protected by a privacy enhancing technology (PET) or security enhancing technology (SET).

Clause 11. The method of claim 1, wherein the PET or SET involves encryption of the file.

Clause 12. The method of claim 11, further comprising:

identifying, by the device, a key associated with the encryption; and

decrypting, by the device via at least the identified key, the encrypted file.

Clause 13. The method of claim 1, wherein the request further comprises information identifying a number of users required to perform the real-world task, wherein the selection of the user is based on the number of users, the search query further comprises the information identifying the number of users.

Clause 14. The method of claim 1, wherein the user profiles in the database correspond at least to a department of the military, wherein each user profile is associated with a user of the military.

Clause 15. A non-transitory computer-readable storage medium tangibly encoded with computer-executable instructions, that when executed by a device, perform a method comprising steps of:

receiving, by the device, a request associated with a real-world task, the request comprising an electronic file, the electronic file comprising information related to the real-world task, the real-world task comprising a set of activities that are required to be completed;

parsing, by the device, the request;

identifying, by the device, based on the parsing of the request, the file;

analyzing, by the device, the file, and determining criteria associated with each of the set of activities, each determined criterion corresponding to a required characteristic a user must possess to complete a respective activity associated with the real-world task;

compiling, by the device, a search query based on the determined criteria;

searching, by the device, a database based on the search query, the database comprising a plurality of user profiles, each user profile comprising characteristics of a user;

determining, by the device, based on the search, a search result identifying a set of user profiles, each identified user profile comprising characteristics complying with the determined criteria;

ranking, by the device, the set of user profiles identified within the search result by ordering each identified user profile according to a measure of a respective user profile’s compliance with the determined criteria;

selecting from the ranked search result, by the device, a user profile; and

securely transmitting, via the device, data associated with the real-world task to an account of the user associated with the selected user profile.

Clause 16. The non-transitory computer-readable storage medium of claim 15, wherein the transmission of the data enables the user to access at least one of classified and protected information related to the real-world task and to equipment for performing the real-world task.

Clause 17. The non-transitory computer-readable storage medium of claim 15, wherein the searching of the database further comprises:

based on the determined criteria in the search query, analyzing, by the device, each user profile in the database;

determining, by the device, a mission score for each user profile, the mission score providing a measure of the user’s complying characteristics; and

based on the determined mission scores, generating, by the device, the ranked search result.

Clause 18. The non-transitory computer-readable storage medium of claim 17, wherein each user profile in the ranked search result has a mission score satisfying a mission threshold corresponding to a minimum set of characteristics.

Clause 19. The non-transitory computer-readable storage medium of claim 15, further comprising:

identifying, by the device, based on the search, another set of user profiles, each identified user profile in the other set corresponds to a user profile with characteristics satisfying at least one of the determined criteria associated with the set of activities encompassing the real-world task.

Clause 20. The non-transitory computer-readable storage medium of claim 19, further comprising:

determining a prospective team of users based on the identified set of other user profiles; and

selecting, from the prospective team, an actual team to perform the real-world task, wherein the secure data is made available to each member of the actual team.

Clause 21. The non-transitory computer-readable storage medium of claim 20, further comprising:

determining, by the device, which members of the actual team are to be associated with performing each activity of the set of activities of the real-world task;

partitioning according to each activity, by the device, the data; and

sending each member of the actual team a respective partitioned portion of the data.

Clause 22. The non-transitory computer-readable storage medium of claim 15, wherein the characteristics of the user in the user profile are selected from a group of information related to the user consisting of: a personal or other identifier, demographic information, geographic information, behavioral history, history of task completion, rank, military unit, as association with the United States’ Department of Defense (DOD), an association with another country’s governmental organization responsible for defense of the country, biometric information, pain tolerance information, treatment plan information, training metrics, psychological information, intelligence quotient (IQ) scores, emotional quotient (EQ) scores, classification testing scores and user-provided feedback.

Clause 23. The non-transitory computer-readable storage medium of claim 15, further comprising:

analyzing, by the device via a classifier model, the search result; and

automatically selecting, without user input, by the device, the user profile.

Clause 24. The non-transitory computer-readable storage medium of claim 15, wherein the file included within the request is protected by a privacy enhancing technology (PET) or security enhancing technology (SET).

Clause 25. The non-transitory computer-readable storage medium of claim 15, wherein the PET or SET involves encryption of the file.

Clause 26. The non-transitory computer-readable storage medium of claim 25, further comprising:

identifying, by the device, a key associated with the encryption; and

decrypting, by the device via at least the identified key, the encrypted file.

Clause 27. The non-transitory computer-readable storage medium of claim 15, wherein the request further comprises information identifying a number of users required to perform the real-world task, wherein the selection of the user is based on the number of users, the search query further comprises the information identifying the number of users.

Clause 28. The non-transitory computer-readable storage medium of claim 15, wherein the user profiles in the database correspond at least to a department of the military, wherein each user profile is associated with a user of the military.

Clause 29. A device comprising:

a processor configured to:

receive a request associated with a real-world task, the request comprising an electronic file, the electronic file comprising information related to the real-world task, the real-world task comprising a set of activities that are required to be completed;

parse the request;

identify, based on the parsing of the request, the file;

analyze, the file, and determine criteria associated with each of the set of activities, each determined criterion corresponding to a required characteristic a user must possess to complete a respective activity associated with the real-world task;

compile a search query based on the determined criteria;

search a database based on the search query, the database comprising a plurality of user profiles, each user profile comprising characteristics of a user;

determine, based on the search, a search result identifying a set of user profiles, each identified user profile comprising characteristics complying with the determined criteria;

rank the set of user profiles identified within the search result by ordering each identified user profile according to a measure of a respective user profile’s compliance with the determined criteria;

select a user profile from the ranked search result; and

securely transmit data associated with the real-world task to an account of the user associated with the selected user profile.

Clause 30. The device of claim 29, wherein the transmission of the data enables the user to access at least one of classified and protected information related to the real-world task and to equipment for performing the real-world task.

Clause 31. The device of claim 29, wherein the processor is further configured to:

based on the determined criteria in the search query, analyze each user profile in the database;

determine a mission score for each user profile, the mission score providing a measure of the user’s complying characteristics; and

based on the determined mission scores, generate the ranked search result.

Clause 32. The device of claim 29, wherein the processor is further configured to:

identify, based on the search, another set of user profiles, each identified user profile in the other set corresponds to a user profile with characteristics satisfying at least one of the determined criteria associated with the set of activities encompassing the real-world task.

Clause 33. The device of claim 32, wherein the processor is further configured to:

determine a prospective team of users based on the identified set of other user profiles; and

select, from the prospective team, an actual team to perform the real-world task, wherein the secure data is made available to each member of the actual team.

Clause 34. The device of claim 33, wherein the processor is further configured to:

determine which members of the actual team are to be associated with performing each activity of the set of activities of the real-world task;

partition, according to each activity, the data; and

send each member of the actual team a respective partitioned portion of the data.

Clause 35. The device of claim 29, wherein the processor is further configured to:

analyze, via a classifier model, the search result; and

automatically select, without user input, the user profile.

Clause 36. The device of claim 29, wherein the file included within the request is protected by a privacy enhancing technology (PET) or security enhancing technology (SET), wherein the PET or SET involves encryption of the file.

Clause 37. The device of claim 36, wherein the processor is further configured to:

identify a key associated with the encryption; and

decrypt, via at least the identified key, the encrypted file.

Clause 38. The device of claim 29, wherein the request further comprises information identifying a number of users required to perform the real-world task, wherein the selection of the user is based on the number of users, the search query further comprises the information identifying the number of users.

For the purposes of this disclosure a module is a software, hardware, or firmware (or combinations thereof) system, process or functionality, or component thereof, that performs or facilitates the processes, features, and/or functions described herein (with or without human interaction or augmentation). A module can include sub-modules. Software components of a module may be stored on a computer readable medium for execution by a processor. Modules may be integral to one or more servers, or be loaded and executed by one or more servers. One or more modules may be grouped into an engine or an application.

For the purposes of this disclosure the term “user”, “patient”, “soldier”, “subscriber” “consumer” or “customer” should be understood to refer to a user of an application or applications as described herein and/or a consumer of data supplied by a data provider. By way of example, and not limitation, the term “user” or “subscriber” can refer to a person who receives data provided by the data or service provider over the Internet in a browser session, or can refer to an automated software application which receives the data and stores or processes the data.

Those skilled in the art will recognize that the methods and systems of the present disclosure may be implemented in many manners and as such are not to be limited by the foregoing exemplary embodiments and examples. In other words, functional elements being performed by single or multiple components, in various combinations of hardware and software or firmware, and individual functions, may be distributed among software applications at either the client level or server level or both. In this regard, any number of the features of the different embodiments described herein may be combined into single or multiple embodiments, and alternate embodiments having fewer than, or more than, all of the features described herein are possible.

Functionality may also be, in whole or in part, distributed among multiple components, in manners now known or to become known. Thus, myriad software/hardware/firmware combinations are possible in achieving the functions, features, interfaces and preferences described herein. Moreover, the scope of the present disclosure covers conventionally known manners for carrying out the described features and functions and interfaces, as well as those variations and modifications that may be made to the hardware or software or firmware components described herein as would be understood by those skilled in the art now and hereafter.

Furthermore, the embodiments of methods presented and described as flowcharts in this disclosure are provided by way of example in order to provide a more complete understanding of the technology. The disclosed methods are not limited to the operations and logical flow presented herein. Alternative embodiments are contemplated in which the order of the various operations is altered and in which sub-operations described as being part of a larger operation are performed independently.

While various embodiments have been described for purposes of this disclosure, such embodiments should not be deemed to limit the teaching of this disclosure to those embodiments. Various changes and modifications may be made to the elements and operations described above to obtain a result that remains within the scope of the systems and processes described in this disclosure.

What is claimed is:

1. A method comprising the steps of:
receiving, by a device, a request to determine a probability that a user is capable of performing a real-world task;
identifying, by the device, a user profile associated with the user, the user profile comprising characteristics of the user that indicate a skill-set of the user;
based on at least one of i) at least a portion of the characteristics of the user and ii) the real-world task, determining, via a predictive model, a predicted performance of the user in the real-world task;
determining, by the device, an output indicating the predicted performance of the user in the real-world task, the output comprising information related to a status of the real-world task and a performance level associated with the status;
based on the output, generating, using one or more computer-implemented models, a treatment plan comprising at least a minimum range of motion; and
automatically controlling, using the treatment plan, operation of at least one operating parameter of at least one physical portion of an electromechanical machine, wherein the operating parameter pertains to the at least minimum range of motion enabled by the at least one physical portion and to a force on the at least one physical portion, wherein the force is computable based on at least the at least minimum range of motion.

2. The method of claim 1, wherein the real-world task comprises a set of activities that are required to be completed for the real-world task to be considered complete, and the status of the real-world task corresponds to an indication as to whether all or at least a portion of the set of activities are determined as capable of being performed by the user, wherein the performance level corresponds to a metric associated with the predicted performance of the user.

3. The method of claim 1, further comprising:
presenting, by the device, a set of tasks to the user;
receiving, by the device, a set of results for the user, the set of results providing an indication related to success of the user in completing each task in the set of tasks;
analyzing, by the device, the set of results; and
determining, by the device, based on the analysis, a value related to the indication of completion of each task in the set of tasks.

4. The method of claim 3, further comprising:
analyzing, by the device, via a machine learning (ML) algorithm, the set of results; and
determining, by the device, based on the ML analysis, information related to performance of the user, the information associated with one or more of the tasks in the set of tasks, wherein the performance information corresponds to the determined value.

5. The method of claim 3, wherein at least a portion of the characteristics in the user profile corresponds to the determined value.

6. The method of claim 3, wherein the set of tasks comprises at least one of a physical activity, a digital activity, and a physical activity and digital activity.

7. The method of claim 3, wherein the set of tasks corresponds at least to the treatment plan, wherein the set of results provided for the user corresponds to a measure of whether the user complied with the treatment plan.

8. The method of claim 1, wherein the characteristics of the user in the user profile are selected from a group of information related to the user consisting of: a personal or other identifier, demographic information, geographic information, behavioral history, history of task completion, rank, military unit, as association with the United States’ Department of Defense (DOD), an association with another country’s governmental organization responsible for defense of the country, biometric information, pain tolerance information, treatment plan information, training metrics, psychological information, intelligence quotient (IQ) scores, emotional quotient (EQ) scores, classification testing scores and user-provided feedback.

9. The method of claim 1, wherein the skill-set of the user corresponds to at least one of a set of physical capabilities, a set of intellectual capabilities, and a set of emotional capabilities.

10. The method of claim 1, wherein the predictive model performing the analysis of the characteristics in the user profile is defined by at least one of a machine learning (ML) algorithm and artificial intelligence (AI) algorithm.

11. The method of claim 1, further comprising:
storing, by the device, in an associated database, information related to the output, the storing comprising updating the user profile; and
generating, by the device, a graphical display of the output.

12. The method of claim 1, further comprising:
analyzing, by the device, the user profile of the user, and determining, based on the analysis of the user profile, a metric indicating a current performance level of the user;
comparing, by the device, the determined metric to a threshold that corresponds to a baseline indicator for performing the real-world task;
determining, by the device, based on the comparison, that the determined metric does not satisfy the threshold; and
generating, by the device, the treatment plan for user, the treatment plan related to improving performance of the real-world task to at least a threshold satisfying level.

13. The method of claim 1, wherein the real-world task corresponds to a military operation, the military operation requiring a set of capabilities operable at a level surpassing a threshold associated with civilian operations.

14. The method of claim 1, further comprising:
identifying a plurality of users, wherein the steps of the method are performed for the plurality of users.

15. The method of claim 1, further comprising:
causing the device to transmit, over a network, information related to at least the user and the output to a third-party platform, the transmission requesting supplemental content related to the information;
receiving, by the device over the network, the supplemental content, the supplemental content being one or more items of electronic content; and
causing, by the device, in connection with the transmitted output, the supplemental content to be displayed.

16. A non-transitory computer-readable storage medium tangibly encoded with computer-executable instructions, that when executed by a device, perform a method comprising steps of:
receiving, by the device, a request to determine a probability that a user is capable of performing a real-world task;
identifying, by the device, a user profile associated with the user, the user profile comprising characteristics of the user that indicate a skill-set of the user;
based on at least one of i) at least a portion of the characteristics of the user and ii) the real-world task, determining, via a predictive model, a predicted performance of the user in the real-world task;
determining, by the device, an output indicating the predicted performance of the user in the real-world task, the output comprising information related to a status of the real-world task and a performance level associated with the status;
based on the output, generating, using one or more computer-implemented models, a treatment plan comprising at least a minimum range of motion; and
automatically controlling, using the treatment plan, operation of at least one operating parameter at least physical portion of an electromechanical machine, wherein the operating parameter pertains to the at least minimum range of motion enabled by the at least one physical portion and to a force on the at least one physical portion, wherein the force is computable based on at least the at least minimum range of motion.

17. The non-transitory computer-readable storage medium of claim 16, wherein the real-world task comprises a set of activities that are required to be completed for the real-world task to be considered complete, and the status of the real-world task corresponds to an indication as to whether all or at least a portion of the set of activities are determined as capable of being performed by the user, wherein the performance level corresponds to a metric associated with the expected performance of the user.

18. The non-transitory computer-readable storage medium of claim 16, further comprising:
presenting, by the device, a set of tasks to the user;
receiving, by the device, a set of results for the user, the set of results providing an indication related to success of the user in completing each task in the set of tasks; and
analyzing, by the device, the set of results, and determining, based on the analysis, a value related to the indication of completion of each task in the set of tasks.

19. The non-transitory computer-readable storage medium of claim 18, further comprising:
analyzing, by the device, via a machine learning (ML) algorithm, the set of results; and
determining, by the device, based on the ML analysis, information related to performance of the user, the information is associated with one or more of the tasks in the set of tasks, wherein the performance information corresponds to the determined value.

20. A device comprising:
a processor configured to:
receive a request to determine a probability that a user is capable of performing a real-world task;
identify a user profile associated with the user, the user profile comprising characteristics of the user that indicate a skill-set of the user;
based on at least one of i) at least a portion of the characteristics of the user and ii) the real-world task, determine, via a predictive model, a predicted performance of the user in the real-world task;
determine an output indicating the predicted performance of the user in the real-world task, the output comprising information related to a status of the real-world task and a performance level associated with the status;
based on the output, generate, using one or more computer-implemented models, a treatment plan comprising at least a minimum range of motion; and
automatically control, using the treatment plan, operation of at least one operating parameter of at least one physical portion of an electromechanical machine, wherein the operating parameter pertains to the at least minimum range of motion enabled by the at least one physical portion and to a force on the at least one physical portion, wherein the force is computable based on at least the at least minimum range of motion.
